(19) United States
(12) Patent Application Publication
(10) Pub. No.: US 2025/0243169 A1
(43) Pub. Date: Jul. 31, 2025

(54) PROCESS FOR THE PREPARATION OF DIVERSELY SUBSTITUTED CYANOCYCLOPROPYL-HETEROCYCLES OR ARENES

(71) Applicant: Syngenta Crop Protection AG, Basel (CH)

(72) Inventors: Raphael Demeunier, Stein (CH); Renaud Beaudegnies, Stein (CH); Martin Pouliot, Stein (CH); Sujit Jumar GHORAI, Corlim, IIhas (IN)

(73) Assignee: Syngenta Crop Protection AG, Basel (CH)

(21) Appl. No.: 18/854,308

(22) PCT Filed: Apr. 5, 2023
(86) PCT No.: PCT/EP2023/059045

§ 371 Date: Oct. 7, 2024

(30) Foreign Application Priority Data
Apr. 7, 2022 (IN) 202211020950

(51) Int. Cl. C07D 239/38; C07C 255/51; C07C 323/32; C07D 213/57; C07D 213/85; C07D 231/14; C07D 237/08; C07D 237/18; C07D 241/12; C07D 241/42; C07D 241/44; C07D 261/08; C07D 277/36; C07D 277/587; C07D 277/64

(52) U.S. Cl. CPC C07D 239/38; C07C 255/51; C07C 323/32; C07D 213/57; C07D 213/85; C07D 231/14; C07D 237/08; C07D 237/18; C07D 241/12; C07D 241/42; C07D 241/44; C07D 261/08; C07D 277/36; C07D 277/587; C07D 277/64

(57) ABSTRACT

A process for the preparation of compound of formula I is provided:

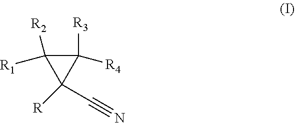

where R, R₁, R₂, R₃ and R₄ are as defined in the description.

The present invention relates to the preparation of diversely substituted cyanocyclopropyl-heteroarenes or arenes that are useful as intermediates for the preparation of active ingredients.

Certain cyanocyclopropyl-heteroarenes or arenes are useful intermediates for the preparation of biologically active compounds as previously described, for example, in: WO16121997, WO20013147 and WO22043468

Known synthesis of such cyanocyclopropyl-heteroarenes from Halo-heteroarenes involve many reaction steps or require the use of expensive transition metals. Most often, those steps are first, replacement of the halogen or sulfone by alkyl-2-cyanoacetate in a nucleophilic aromatic substitution, followed by saponification of the ester, decarboxylation of the acid thus obtained, and finally cyclopropanation with 1,2-di-halo-ethane and a base. Alternatively, when the Leaving Group is a halogen activated enough for oxidative insertion by a transition metal-based catalyst such as [Pd-Lₙ], direct coupling with the anion of cycanocyclopropyl may be considered. Both approaches are shown in Scheme 1 (A=C or N; LG=Leaving Group, such as halogen or sulfone).

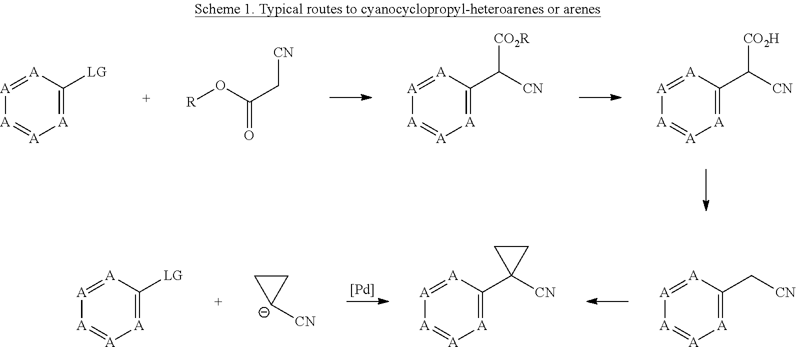

Such long and laborious syntheses are not optimal for preparing large amount of material due to low overall yields and large amount of waste generated, or to the use of expensive noble metal catalysts. Therefore, it would be advantageous to have available a more efficient and economical route to these useful intermediates.

The present invention therefore provides a process for the preparation of cyanocyclopropyl-arenes or heteroarenes of formula (I)

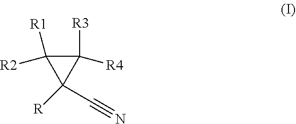

wherein R is selected from Rₐ or R_{b}, wherein Rₐ is aryl substituted by at least one electron withdrawing group; R_{b} is an unsubstituted or substituted heteroaryl and R₁, R₂, R₃, and R₄ are, independently from each other, hydrogen, alkyl, aryl, aryloxyalkyl or haloaryloxyalkyl, wherein at least one of R₃ or R₄ is hydrogen.

In accordance with a first aspect of the present invention, the direct replacement of a halogen atom or a sulfone group in electrophilic, activated positions of arenes (Rₐ) or heteroarenes (R_{b}) of formula (II) and (III), using a base and a reagent of formula (IV)-a yields in a single step a compound of formula (I)-a wherein R is defined as previously and LG is a leaving group constituted of a halogen atom or a sulfone group (Scheme 2).

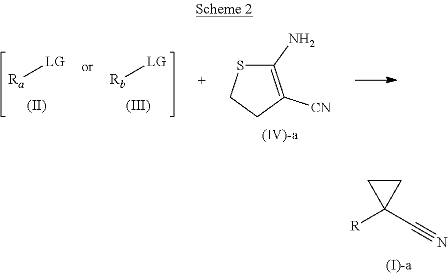

The compound of formula (IV)-a is known, commercially available and its synthesis described by Yamagata, Kenji et al in Chemical & Pharmaceutical Bulletin, 30(12), 4396-401; 1982; or by Wamhoff, Heinrich and Thiemig, Heinz Albrecht in Chemische Berichte, 118(11), 4473-85; 1985.

Aryl and alkyl substituents can be introduced by using more elaborated reagents of formula (IV) wherein R₁, R₂, R₃, and R₄ are as defined above (Scheme 3),

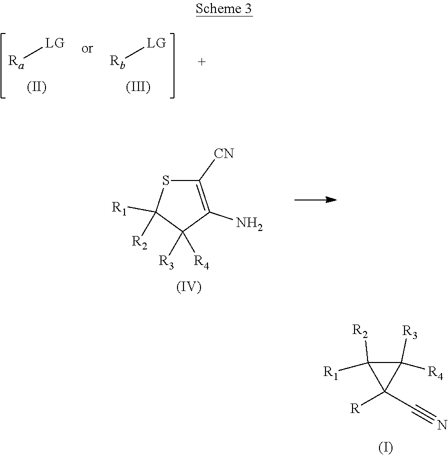

The compounds of formula (IV) are prepared by the same way as compound of formula (IV)-a, but by using substituted thiiranes of formula VI) (wherein R₁, R₂, R₃, and R₄ are as defined above)

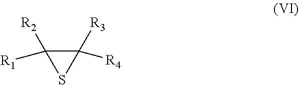

which are made simply from corresponding epoxides and potassium thiocyanate (KSCN) (Scheme 4),

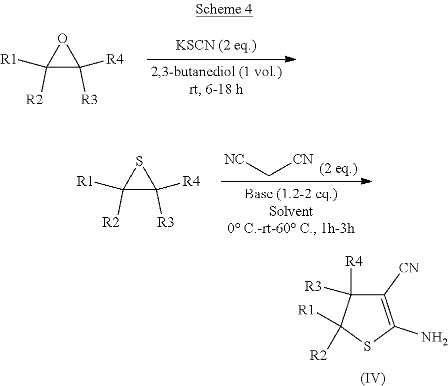

as described by Naoto Aoyagi et al in Chemistry Select, 2, 4466-4468; 2017; followed by treatment with malonitrile and a base as NaH as described in Yamagata, Kenji et al in Chemical & Pharmaceutical Bulletin, 30(12), 4396-401; 1982; (NMP as solvent instead of DMSO for safety reasons) or by LiOH·H₂O (in THF or MeTHF as solvent) or alternatively, by NaH in DME as solvent as described in Wamhoff, Heinrich and Thiemig, Heinz Albrecht in Chemische Berichte, 118(11), 4473-85; 1985. In scheme 4, R₁, R₂, R₃, and R₄ are as defined above.

Thus, according to the present invention there is provided a process for the preparation of a compound of formula (I)

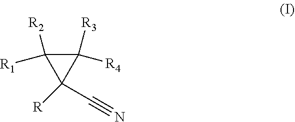

wherein
R is selected from Rₐ or R_{b}, wherein Rₐ is an aryl group substituted by at least one electron withdrawing substituent; R_{b} is an unsubstituted or substituted heteroaryl group and R₁, R₂, R₃, and R₄ are, independently from each other, hydrogen, alkyl, aryl, aryloxyalkyl or haloaryloxyalkyl, wherein at least one of R₃ or R₄ is hydrogen; which process comprises:
reacting a compound of formula (II) or (III)

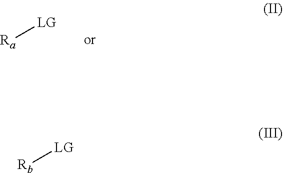

wherein Rₐ and R_{b} are as defined in formula (I); and LG is a halogen or a sulfone group, with a compound of formula (IV),

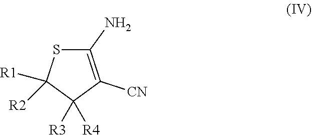

wherein R₁, R₂, R₃, and R₄ are as defined in formula (I);
in the presence of a suitable base, in an appropriate solvent (or diluent);
to produce a compound of formula (I)

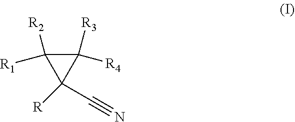

wherein R, R₁, R₂, R₃, and R₄ are as defined under formula I above.

In one embodiment, the present invention relates to a process for the preparation of a compound of formula (I) as described above, with the exception of a process for the preparation of 5-(1-cyanocyclopropyl)-pyridine-2-carboxylic acids, esters, amides and nitriles of formula I-b or agrochemically acceptable salts thereof

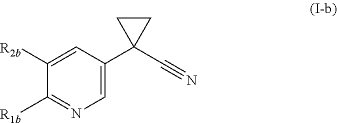

wherein R_{1b} is —CO₂R_{4b}, —CO(NR_{5b}R_{6b}), carboxylate or cyano; R₂ₐ is hydrogen, halogen or —SR_{3b}; R_{3b} is C₁-C₄alkyl or C₃-C₆cycloalkyl-C₁-C₄alkyl; R_{4b} is hydrogen, —Si(CH₃)₃ or C₁-C₆alkyl; and R_{5b} and R_{6b} are, independently from each other, hydrogen or C₁-C₄alkyl.

A second aspect of the present invention provides certain compounds of formula I represented by the compounds of formula (I)-a

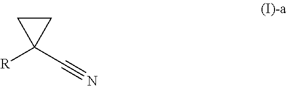

wherein R is as defined in formula (I) above.

In another embodiment, the present invention relates to certain compounds of formula I represented by the compounds of formula (I)-a as described above, with the exception of a compound of formula (I)-a which is a 5-(1-cyanocyclopropyl)-pyridine-2-carboxylic acid, ester, amide or nitrile of formula I-b or an agrochemically acceptable salt thereof

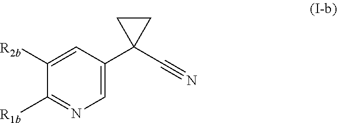

wherein R_{1b} is —CO₂R_{4b}, —CO(NR_{5b}R_{6b}), carboxylate or cyano; R₂ₐ is hydrogen, halogen or —SR_{3b}; R_{3b} is C₁-C₄alkyl or C₃-C₆cycloalkyl-C₁-C₄alkyl; R_{4b} is hydrogen, —Si(CH₃)₃ or C₁-C₆alkyl; and R_{5b} and R_{6b} are, independently from each other, hydrogen or C₁-C₄alkyl.

The process of the present invention is demonstrated to be of great usefulness as it allows the synthesis of key building blocks for the preparation of active ingredients in higher yields and with more favorable conditions with respect to previously described routes.

Additional aspects of the process according to the invention for making compounds of formula (I) are further detailed and explained by reference to scheme 5, wherein R, LG, R₁ to R₄ are defined as above. Some intermediates compounds are made and further transformed in the process of making compounds of formula (I), which can then be detailed as passing through the following intermediates in a single pot operation (Scheme 5)

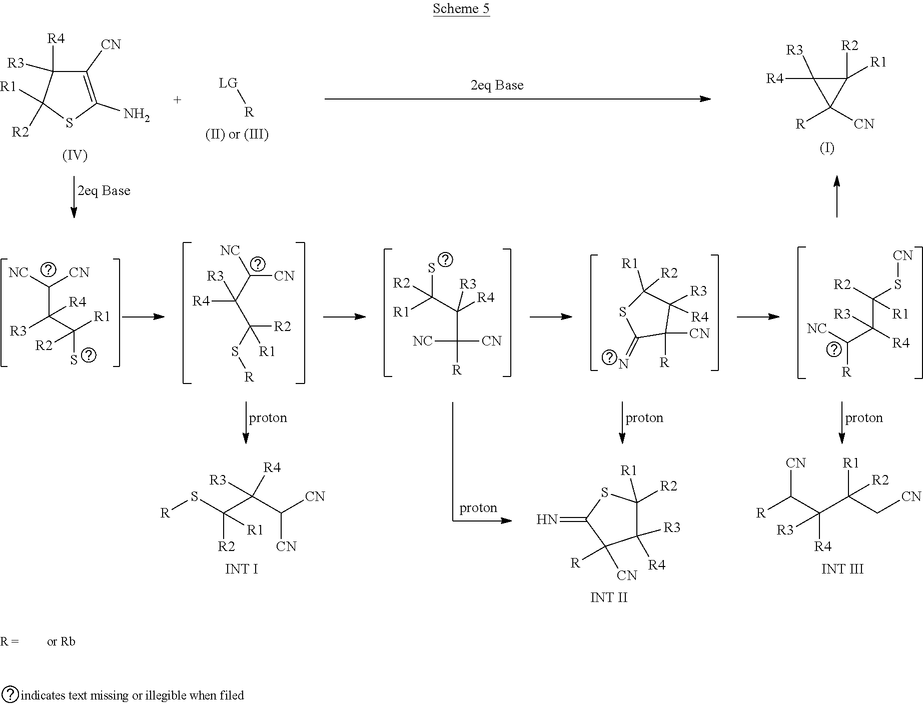

The intermediate salts can sometimes be protonated, isolated and characterized under the forms of INT I, INT II, or INT III before their complete transformations into compounds of formula (I). In this regard, the compounds INT I, INT II, or INT III can also be resubmitted to basic conditions in a separate, subsequent step to provide compounds of formula (I). In the process according to the invention for making compounds of formula (I), examples of suitable bases are alkali metal hexamethyldisilazides, alkaline earth metal hexamethyldisilazides, alkali metal hydroxides, alkali metal alkoxides or alkaline earth metal alkoxides. Examples which may be mentioned are sodium hydroxide, potassium hydroxide, sodium methanolate, sodium tertiobutanolate, and potassium tertiobutanolate; preferably an alkali metal hydroxide, more preferably sodium hydroxide.

In the process according to the invention of making compounds of formula (I), appropriate solvents (or diluents) are polar solvents, such as dimethylformamide, dimethylsulfoxide, N-methyl-pyrrolidine, dimethylacetamide, sulfolane, N,N′-dimethylpropyleneurea (DMPU); more preferably polar organic solvents chosen from dimethylformamide, dimethylsulfoxide, N-methyl-pyrrolidine.

In one embodiment, in the process according to the invention of making compounds of formula (I), the reaction is advantageously carried out in a temperature range from approximately 0° C. to approximately +100° C., preferably from approximately +20° C. to approximately +80° C.

Another preferred embodiment of the process for the preparation of compounds of formula I as shown and explained in scheme 5 is further detailed by reference to scheme 6 which shows specific embodiments of the formulae I, III, INT I, INT II and INT III as represented by compounds of formulae (I)-a, (IV)-a, INT Ia, INT IIa and INT IIIa, respectively.

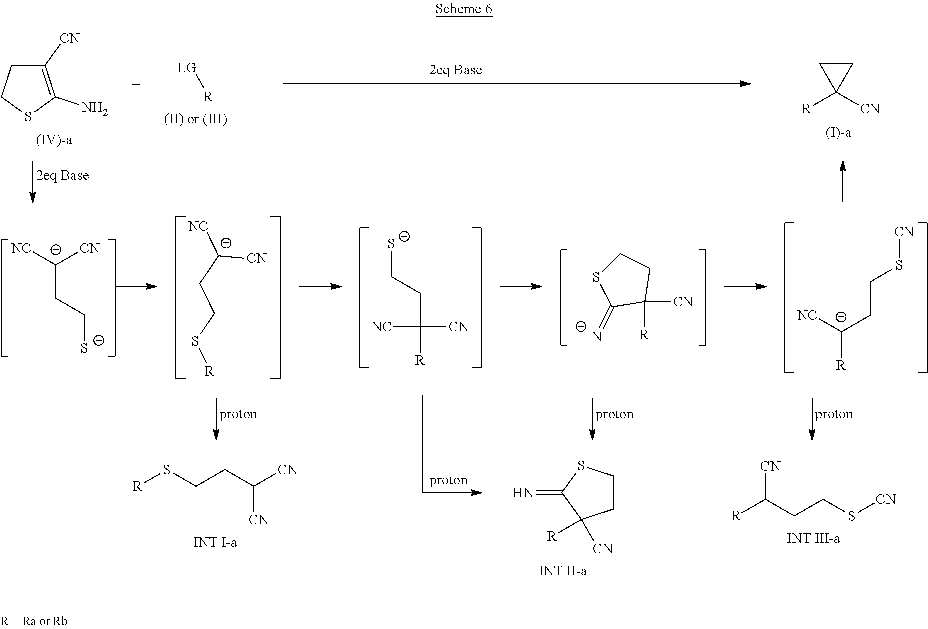

In one embodiment, the anions of INT I are intermediates which often do accumulate to a reasonable extent during the course of the reaction, so that if the reaction is interrupted before completion, INT I can be isolated and purified, as can be seen in the preparatory examples. In particular, if the first steps to anions of INT I are run at lower temperature (room temperature or even −10° C. to −20° C.), a good yield of INT I can be achieved, if it is wished to interrupt the reaction at this stage.

In another embodiment, anions of INT II and INT III do not accumulate to significant levels in the reaction mixture. In some cases, they can be observed as anions by in situ monitoring or (as their protonated forms) by LCMS of reactions samples, but their amounts are too low to be able to interrupt the reaction, isolate and characterize pure samples of INT II or INT III.

In another aspect, the present invention also relates to compounds of the formula INT I

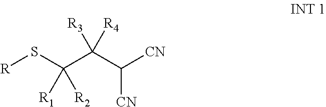

wherein R, R₁, R₂, R₃, and R₄ are defined as under formula I above.

In one embodiment, the present invention also relates to compounds of the formula INT I as described above, with the exception of a compound of formula INT Ib

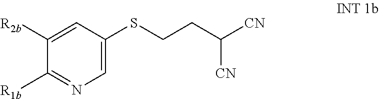

wherein R_{1b} is —CO₂R_{4b}, —CO(NR_{5b}R_{6b}), carboxylate or cyano; R_{2b} is hydrogen, halogen or —SR_{3b}; R_{3b} is C₁-C₄alkyl or C₃-C₆cycloalkyl-C₁-C₄alkyl; R_{4b} is hydrogen, —Si(CH₃)₃ or C₁-C₆alkyl; and R_{5b} and R_{6b} are, independently from each other, hydrogen or C₁-C₄alkyl.

Certain preferred embodiments according to the invention are provided as set out below.

Embodiment 1 provides a process for preparing compounds of formula I as defined above.

Preferably embodiment 1 relates to a process for the preparation of a compound of formula (I) as described above, with the exception of a process for the preparation of 5-(1-cyanocyclopropyl)-pyridine-2-carboxylic acids, esters, amides and nitriles of formula I-b or agrochemically acceptable salts thereof as described above.

Embodiment 2 provides a process for the preparation of a compound of formula (I) which process comprises: reacting a compound of formula (II) or (III) with a compound of formula (IV), to produce a compound of formula (I) as defined above.

Preferably embodiment 2 relates to a process for the preparation of a compound of formula (I) as described above, with the exception of a process for the preparation of 5-(1-cyanocyclopropyl)-pyridine-2-carboxylic acids, esters, amides and nitriles of formula I-b or agrochemically acceptable salts thereof as described above.

Embodiment 3 provides a process for the preparation of a compound of formula I-a which process comprises: reacting a compound of formula (II) or (III) with a compound of formula (IV)-a, to produce a compound of formula I-a as provided in scheme 2 as defined above.

Preferably embodiment 3 relates to a process for the preparation of a compound of formula (I-a) as described above, with the exception of a process for the preparation of 5-(1-cyanocyclopropyl)-pyridine-2-carboxylic acids, esters, amides and nitriles of formula I-b or agrochemically acceptable salts thereof as described above.

Embodiment 4 provides a process for the preparation of a compound of formula (I) which process comprises: reacting a compound of formula (II) or (111) with a compound of formula (IV), to produce a compound of formula (I) as provided in scheme 3 as defined above.

Preferably embodiment 4 relates to a process for the preparation of a compound of formula (I) as described above, with the exception of a process for the preparation of 5-(1-cyanocyclopropyl)-pyridine-2-carboxylic acids, esters, amides and nitriles of formula I-b or agrochemically acceptable salts thereof as described above.

Embodiment 5 provides a process for the preparation of a compound of formula (I) which process comprises: reacting a compound of formula (II) or (III) with a compound of formula (IV), to produce a compound of formula (I) as provided in scheme 5 as defined above.

Preferably embodiment 5 relates to a process for the preparation of a compound of formula (I) as described above, with the exception of a process for the preparation of 5-(1-cyanocyclopropyl)-pyridine-2-carboxylic acids, esters, amides and nitriles of formula I-b or agrochemically acceptable salts thereof as described above.

Embodiment 6 provides a process for the preparation of a compound of formula I-a which process comprises: reacting a compound of formula (II) or (III) with a compound of formula (IV)-a, to produce a compound of formula I-a as provided in scheme 6 as defined above.

Preferably embodiment 6 relates to a process for the preparation of a compound of formula (I-a) as described above, with the exception of a process for the preparation of 5-(1-cyanocyclopropyl)-pyridine-2-carboxylic acids, esters, amides and nitriles of formula I-b or agrochemically acceptable salts thereof as described above.

Embodiment 7 provides a compound of formula I-a as defined above.

Preferably embodiment 7 relates to a compound of formula (I-a) as described above, with the exception of compounds that are 5-(1-cyanocyclopropyl)-pyridine-2-carboxylic acids, esters, amides and nitriles of formula I-b or agrochemically acceptable salts thereof as described above Embodiment 8 provides a compound of formula INT I as defined above Preferably embodiment 8 relates to a compound of formula INT I as described above, with the exception of a compound of formula INT Ib as described above.

With respect to embodiments 1-8, preferred values of R, R₁, R₂, R₃, and R₄ are, in any combination thereof, as set out below:

Preferably R is selected from Rₐ or R_{b}, wherein Rₐ is aryl substituted by at least one electron withdrawing group; R_{b} is an unsubstituted or substituted heteroaryl.

Preferably Rₐ is a carbocyclic aromatic ring system (such as phenyl or naphthyl) that is mono- or polysubstituted (preferably mono- or di-substituted) by substituents selected from the group consisting of halogen (such as chloro, bromo), cyano, alkyl, haloalkyl, nitro, phenyl, halophenyl, esters, ketones, amides; and wherein at least one such substituent is an electron withdrawing substituent selected from CF₃, NO₂, CN, esters, ketones, and amides. In one embodiment, Ra is phenyl which is mono- or di-substituted by an electron withdrawing substituent selected from CF₃, NO₂, CN, esters, ketones, and amides.

Preferably R_{b} is is a five- to ten-membered heteroaromatic ring system that is unsubstituted or is mono- or polysubstituted by substituents selected from the group consisting of halogen, cyano, C₁-C₆alkyl, C₁-C₆haloalkyl, nitro, phenyl and halophenyl; and wherein said ring system can contain 1 or more ring heteroatoms selected from the group consisting of nitrogen, oxygen and sulphur.

In one embodiment, R_{b} is pyrimidyl, benzothiazolyl, thiazolyl, pyridyl, pyridazinyl, isoxazolyl, pyrazolyl, quinoxalinyl, or pyrazinyl that is unsubstituted or is mono- or di-substituted by substituents selected from the group consisting of halogen, cyano, C₁-C₇alkyl, C₁-C₇haloalkyl, nitro, phenyl and halophenyl

Preferably R₁, R₂, R₃, and R₄ are, independently from each other, hydrogen, alkyl, aryl, aryloxyalkyl or haloaryloxyalkyl, wherein at least one of R₃ or R₄ is hydrogen.

Also preferred is when R₁, R₂, R₃, and R₄ are, independently from each other, hydrogen, C₁-C₇alkyl, phenyl, phenyloxymethyl or halophenyloxymethyl, wherein at least one of R₃ or R₄ is hydrogen.

More preferably R₁, R₂, R₃, and R₄ are each hydrogen.

Alkyl groups preferably have a chain length of from 1 to 7 carbon atoms and refers to a saturated straight-chain or branched hydrocarbon radical attached via any of the carbon atoms. For example, any one of the radicals methyl, ethyl, n-propyl, 1-methylbutyl, 2-methylbutyl, 3-methylbutyl, 2,2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, or heptyl.

Haloalkyl groups preferably have a chain length of from 1 to 7 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl

Halogen is generally fluorine, chlorine, bromine or iodine. This also applies, correspondingly, to halogen in combination with other meanings, such as haloalkyl or halophenyl.

The preparatory examples which follow are intended to illustrate the invention and show preferred compounds of formulae I, and INT I that can be used and/or prepared according to the process described above.

### PREPARATORY EXAMPLES

“Mp or m.p.” means melting point in ° C. Purity of starting materials, crudes and products was determined with quantitative ¹H NMR using 1,3,5-trimethoxy benzene as an internal standard.

### Example 1: Preparation of 1-pyrimidin-2-ylcyclopropanecarbonitrile

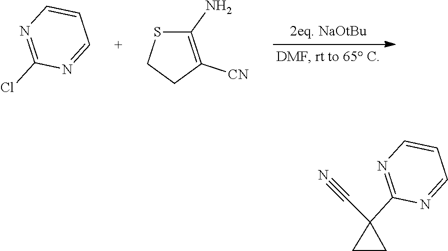 [SMILES: Clc1ncccn1 ; N#CC1=C(N)SCC1 ; N#CC1(c2ncccn2)CC1]

To a solution of 5-amino-2,3-dihydrothiophene-4-carbonitrile (100 mg, 0.79 mmol) and 2-chloropyrimidine (1 equiv., 0.79 mmol) in 3.2 mL DMF were added, at room temperature, 2 equivalents of sodium tert-butanolate (1.58 mmol). After one hour stirring at room temperature, when only the intermediate 2-(2-pyrimidin-2-ylsulfanylethyl)propanedinitrile was observed, the temperature was increased to 65° C. during 3 hours, after which time the reaction mixture was allowed to cool down to room temperature. It was then poured onto 10 mL of saturated, aqueous NH₄Cl solution and stirred for 10 minutes. The resulting mixture was then acidified with 10 mL of aqueous 1N HCl and extracted four times with 10 mL of dichloromethane The combined organic layers were dried over solid MgSO₄, filtered and the solvent was removed under reduced pressure to deliver 231 mg of the crude desired product as an orange solid. The crude was purified using a 12 g silica chromatography column with a gradient of Cyclohexane/Ethyl acetate (up to 1:1) to give 81 mg of the pure, desired product 1-pyrimidin-2-ylcyclopropanecarbonitrile (Isolated Yield 77%).

Alternatively, the 1-pyrimidin-2-ylcyclopropanecarbonitrile can be obtained from isolated 2-(2-pyrimidin-2-ylsulfanylethyl)propanedinitrile (example 2) by following the protocol below.

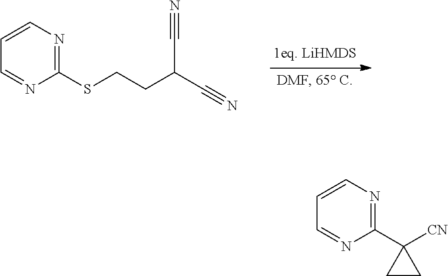 [SMILES: N#CC(C#N)CCSc1ncccn1 ; N#CC1(c2ncccn2)CC1]

To a solution of 2-(2-pyrimidin-2-ylsulfanylethyl)propanedinitrile (50 mg, 0.245 mmol) in 1 mL of DMF at room temperature were added 0.25 mL of a solution of Lithium bis(trimethylsilyl)amide (LiHMDS) 1N in THF. The reaction mixture was then heated at 65° C. for 5 hours, before being allowed to cool down to room temperature. It was then poured onto 5 mL of a saturated aqueous NH₄Cl solution, stirred for 10 minutes and extracted 3 times with 15 mL of dichloromethane. The combined organic layers were dried over solid Na₂SO₄, filtered and the solvent was removed under reduced pressure to deliver 27.4 mg of the crude desired product as an orange solid. The crude was quantified by Q-NMR to give a strength (w/w) of 89% and therefore the reaction gave a chemical yield of 69%.

m.p.=124.2-125.4° C. with gradual degradation up to 126° C.; ¹H NMR (400 MHz, CDCl₃): δ (ppm)=8.64 (d, J=4.8 Hz, 2H), 7.17 (t, J=5.0 Hz, 1H), 1.89-1.77 (m, 4H); ¹³C NMR (101 MHz, CDCl₃): δ (ppm)=165.4, 157.5, 121.2, 119.4, 20.9, 17.6; IR (ATR, Diamond): v (cm⁻¹)=2922 (w), 2239 (m), 1564 (s), 1429 (vs), 961 (s), 784 (s), 635 (s)

### Example 2. Preparation of 2-(2-pyrimidin-2-ylsulfanylethyl)propanedinitrile

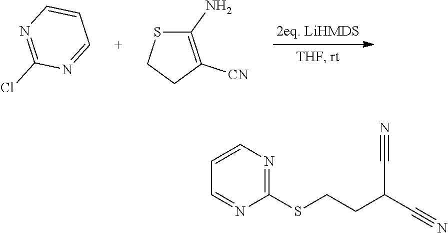 [SMILES: Clc1ncccn1 ; N#CC1=C(N)SCC1 ; N#CC(C#N)CCSc1ncccn1]

To a solution of 5-amino-2,3-dihydrothiophene-4-carbonitrile (200 mg, 1.58 mmol) and 2-chloropyrimidine (1 equiv., 1.58 mmol) in 2.8 mL THF were added, at room temperature, 2 equivalents (3.2 mL) of a solution of Lithium bis(trimethylsilyl)amide (LiHMDS) 1N in THF. After one hour stirring at room temperature, the mixture was poured onto 20 mL of a saturated aqueous NH₄Cl solution and extracted 3 times with 60 mL of dichloromethane. The combined organic layers were washed three times with 20 mL of 1M aq. HCl, then once with 20 mL aq. sat. NaHCO₃, then finally with 20 mL of brine. The combined organic phases were then dried over solid Na₂SO₄, filtered and the solvent was removed under reduced pressure to deliver the crude product 2-(2-pyrimidin-2-ylsulfanylethyl)propanedinitrile as a yellowish solid. The crude was purified using a 12 g silica chromatography column with a gradient of Cyclohexane/Ethyl acetate (up to 1:1) to give 190 mg of the pure, desired product 2-(2-pyrimidin-2-ylsulfanylethyl)propanedinitrile (Isolated Yield 58%) m.p.=65.9-67.8° C.; ¹H NMR (400 MHz, CDCl₃): δ (ppm)=8.55 (d, J=4.8 Hz, 2H), 7.05 (t, J=5.0 Hz, 1H), 4.06 (t, J=7.3 Hz, 1H), 3.38 (t, J=6.6 Hz, 2H), 2.54 (q, J=7.0 Hz, 2H); ¹³C NMR (101 MHz, CDCl₃): δ (ppm)=170.5, 157.8, 117.4, 112.5, 31.1, 27.5, 21.6; IR (ATR, Diamond): v (cm⁻¹)=3038 (vw), 2909 (vw), 2257 (vw), 1701 (w), 1564 (m), 1550 (s), 1380 (vs), 1204 (m), 1186 (m), 774 (m)

### Example 3: Preparation of 1-(1,3-benzothiazol-2-yl)cyclopropanecarbonitrile

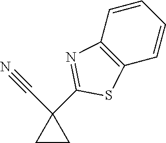 [SMILES: N#CC1(c2nc3ccccc3s2)CC1]

To a solution of 5-amino-2,3-dihydrothiophene-4-carbonitrile (100 mg, 0.79 mmol) and 2-chloro-1,3-benzothiazole (1 equiv., 0.79 mmol) in 3.2 mL DMF were added, at room temperature, 2 equivalents (1.58 mL) of a solution of Sodium bis(trimethylsilyl)amide (NaHMDS) 1N in THF. After four hours stirring at room temperature, the mixture was heated to 65° C. for two hours, after which time it was allowed to cool down to room temperature. It was then poured onto 10 mL of a saturated aqueous NH₄Cl solution and extracted 3 times with 30 mL of dichloromethane. The combined organic layers were washed three times with 10 mL of 1M aq. HCl, then once with 10 mL aq. sat. NaHCO₃, then finally with 10 mL of brine. The combined organic phases were then dried over solid Na₂SO₄, filtered and the solvent was removed under reduced pressure to deliver 145 mg of the crude product 1-(1,3-benzothiazol-2-yl)cyclopropanecarbonitrile as a brownish solid. The crude was quantified by Q-NMR to give a strength (w/w) of 56% and therefore the reaction gave a chemical yield of 51%. The crude was purified using a 12 g silica chromatography column with a gradient of Cyclohexane/Ethyl acetate (up to 1:1) to deliver the pure, desired product 1-(1,3-benzothiazol-2-yl)cyclopropanecarbonitrile. m.p.=139.7-141.5° C.; ¹H NMR (400 MHz, CDCl₃): δ (ppm)=7.91 (d, J=8.1 Hz, 1H), 7.85 (dd, J=8.1, 0.7 Hz, 1H), 7.47 (ddd, J=7.7, 7.7, 1.1 Hz, 1H), 7.43-7.35 (m, 1H), 2.07-2.00 (m, 2H), 1.97-1.90 (m, 2H); ¹³C NMR (101 MHz, CDCl₃): δ (ppm)=166.1, 153.1, 134.9, 126.7, 125.4, 122.9, 121.7, 120.6, 21.7, 14.5; IR (ATR, Diamond): v (cm⁻¹)=3096 (w), 3053 (w), 2247 (m), 1510 (m), 1438 (m), 1279 (s), 1094 (s), 1062 (m), 1048 (s), 762 (vs)

### Example 4: Preparation of 1-(4-chlorothiazol-2-yl)cyclopropanecarbonitrile

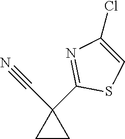 [SMILES: N#CC1(c2nc(Cl)cs2)CC1]

To a solution of 5-amino-2,3-dihydrothiophene-4-carbonitrile (87 mg, 94% w/w, 0.65 mmol) and 2,4-dichlorothiazole (1 equiv., 0.65 mmol) in 5.2 mL DMF were added, at room temperature, 2.2 equivalents of sodium tert-butanolate (1.43 mmol). After three hours stirring at room temperature, the reaction was then poured onto 10 mL of saturated, aqueous NH₄Cl solution and stirred for 10 minutes. The resulting mixture was then acidified with 10 mL of aqueous 1N HCl and extracted three times with 20 mL of ethyl acetate. The combined organic layers were washed with 10 mL aq. sat NaHCO₃, then with 10 mL of brine, and finally dried over solid MgSO₄ and filtered. The solvent was removed under reduced pressure to deliver 122 mg of the crude desired product as a white crystalline solid covered in orange viscous oil. The crude was purified by chromatography column to give 74 mg of the pure, desired product 1-(4-chlorothiazol-2-yl)cyclopropanecarbonitrile (Isolated Yield 62%). m.p.=83.4-85.6° C.; ¹H NMR (400 MHz, CDCl₃): δ (ppm)=7.03 (s, 1H), 1.99-1.85 (m, 4H); ¹³C NMR (101 MHz, CDCl₃): δ (ppm)=165.7, 139.3, 120.1, 113.3, 21.5, 14.3; IR (ATR, Diamond): v (cm⁻¹)=3110 (m), 2243 (m), 1488 (vs), 1285 (s), 1089 (vs), 751 (vs)

### Example 5: Preparation of 1-(4-bromothiazol-2-yl)cyclopropanecarbonitrile

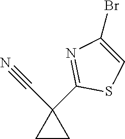 [SMILES: N#CC1(c2nc(Br)cs2)CC1]

To a solution of 5-amino-2,3-dihydrothiophene-4-carbonitrile (520 mg, 4.1 mmol) and 2,4-dibromothiazole (1 g, 1 equiv., 4.1 mmol) in 15 mL DMF were added 2.2 equivalents of sodium tert-butanolate (9.1 mmol). After two hours stirring at room temperature. 5 mL of ice cold water were added and the mixture was acidified with 10 mL of 2N HCl. The aqueous layer was extracted three times with 20 mL of ethyl acetate. The combined organic layers were washed with 10 mL of brine, dried over solid MgSO₄ and filtered. The solvent was removed under reduced pressure to deliver 2.3 g of the crude desired product as an orange liquid, which was quantified by Q-NMR to give a strength (w/w) of 15% in 1-(4-bromothiazol-2-yl)cyclopropanecarbonitrile; therefore the reaction gave a chemical yield of 33% The crude was purified by chromatography column to give 286 mg of the pure, desired product 1-(4-bromothiazol-2-yl)cyclopropanecarbonitrile (Isolated Yield 30%).

Alternatively, the 1-(4-bromothiazol-2-yl)cyclopropanecarbonitrile can be obtained from isolated 2-[2-(4-bromothiazol-2-yl)sulfanylethyl]propanedinitrile (example 6) by following the protocol below. To a solution of 2-[2-(4-bromothiazol-2-yl)sulfanylethyl]propanedinitrile (250 mg, 0.867 mmol) in 5 mL of THF at room temperature were added 0.87 mL of a solution of Lithium bis(trimethylsilyl)amide (LiHMDS) 1N in THF. The reaction mixture was then heated at 65° C. for 2 hours, before being allowed to cool down to room temperature. It was then poured onto 5 mL of a saturated aqueous NH₄Cl solution, stirred for 10 minutes and extracted 3 times with 20 mL of EtOAc The combined organic layers were dried over solid Na₂SO₄, filtered and the solvent was removed under reduced pressure to deliver 254 mg of the crude desired product as an orange liquid. The crude was quantified by Q-NMR to give a strength (w/w) of 67% and therefore the reaction gave a chemical yield of 84%. The crude was purified by chromatography column to give 120 mg of the pure, desired product 1-(4-bromothiazol-2-yl)cyclopropanecarbonitrile as a white solid (Purity measured by Q-NMR being 92% (w/w) the isolated Yield is 56%). ¹H NMR (400 MHz, CDCl₃): δ (ppm)=7.15 (s, 1H), 1.99-1.84 (m, 4H).

### Example 6. Preparation of 2-[2-(4-bromothiazol-2-yl)sulfanylethyl]propanedinitrile

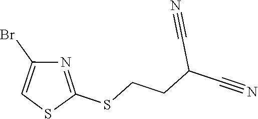 [SMILES: N#CC(C#N)CCSc1nc(Br)cs1]

In a 3 necked round bottom flask equipped with magnetic bar, thermometer and nitrogen inlet was charged 15 mL of DMF, 2,4-dibromothiazole (1.0 g, 4.1 mmol) and 5-amino-2,3-dihydrothiophene-4-carbonitrile (1.0 equiv, 4.1 mmol). The reaction mixture became a light yellow solution. It was cooled to −10° C., then the base sodium tert-butanolate (2.2 equiv., 9.1 mmol, 2M solution in THF) was added at −10° C. for 10 min. (an exotherm of 5° C. was observed). The reaction was carried on at −10° C. for one hour. 5 ml of ice cold water were added and the mixture was acidified with 10 mL of 2N HCl at −5° C. The aqueous layer was extracted thrice with 20 mL of EtOAc, then washed with 20 mL of brine. The combined organic layers were dried over solid Na₂SO₄, filtered and the solvents were removed under reduced pressure. 3.28 grams of an orange oil was obtained, which was quantified by Q-NMR to give a strength (w/w) of 25% in 2-[2-(4-bromothiazol-2-yl)sulfanylethyl]propanedinitrile; therefore the reaction gave a chemical yield of 69%. The crude was purified using a 40 g silica chromatography column with a gradient of Cyclohexane/Ethyl acetate (up to 2:1) to deliver the pure, desired product 2-[2-(4-bromothiazol-2-yl)sulfanylethyl]propanedinitrile as a white solid (0.8 g, Q-NMR purity 96 mass %, isolated yield 65%). ¹H NMR (400 MHz, CDCl₃): δ (ppm)=2.59 (q, J=6.85 Hz, 2H) 3.46 (t, J=6.66 Hz, 2H) 4.16 (t, J=7.46 Hz, 1H) 7.16 (s, 1H)

### Example 7. Preparation of 2-(1-cyanocyclopropyl)pyridine-3-carbonitrile

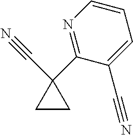 [SMILES: N#Cc1cccnc1C1(C#N)CC1]

To a solution of 5-amino-2,3-dihydrothiophene-4-carbonitrile (94 mg, 94% w/w, 0.7 mmol) and 2-chloropyridine-3-carbonitrile (1 equiv., 0.7 mmol) in 4.9 mL DMF were added, at 0° C., 2.2 equivalents of a 2M sodium tert-butanolate (1.54 mmol) solution in THF. After three hours stirring at 0° C., the reaction was allowed to warm to room temperature and stirred for 4 hours more. It was then poured onto 10 mL of saturated, aqueous NH₄Cl solution and stirred for 10 minutes. The resulting mixture was then acidified with 10 mL of aqueous 1N HCl and extracted three times with 20 mL of ethyl acetate. The combined organic layers were washed with 10 mL aq. sat. NaHCO₃, then with 10 mL of brine (thrice), and finally dried over solid MgSO₄ and filtered. The solvent was removed under reduced pressure to deliver 230 mg of the crude desired product as a yellow oil. The crude was purified by chromatography column to give 90 mg of the pure, desired product 2-(1-cyanocyclopropyl)pyridine-3-carbonitrile (Isolated Yield 76%)

Alternatively, the title product can be made from cyclisation of the isolated intermediate 2-[2-[(3-cyano-2-pyridyl)sulfanyl]ethyl]propanedinitrile (example 8) following this protocol. To a solution of 242-[(3-cyano-2-pyridyl)sulfanyl]ethyl]propanedinitrile (500 mg, 2.19 mmol) in 5 mL of THF at room temperature were added 1.1 mL of the base sodium tert-butanolate (1 equiv., 2.19 mmol. 2M solution in THF). The reaction mixture was then heated at 65° C. for 2 hours, before being allowed to cool down to room temperature. It was then poured onto 5 mL of a saturated aqueous NH₄Cl solution, stirred for 10 minutes and extracted 3 times with 20 mL of EtOAc. The combined organic layers were dried over solid Na₂SO₄, filtered and the solvent was removed under reduced pressure to deliver 440 mg of the crude desired product as an orange liquid. The crude was purified by chromatography column to give 247 mg of the pure, desired product 2-(1-cyanocyclopropyl)pyridine-3-carbonitrile as a white solid (Purity measured by Q-NMR being 96% (w/w) the isolated Yield is 64%). m.p.=74.2-76.6° C.; ¹H NMR (400 MHz, CDCl₃): δ (ppm)=8.71 (dd, J=4.9, 1.64 Hz, 1H), 8.04 (dd, J=7.99, 1.82 Hz, 1H), 7.42 (dd, J=7.99, 5.09 Hz, 1H), 1.90-1.78 (m, 4H); ¹³C NMR (101 MHz, CDCl₃): δ (ppm)=156.3, 152.5, 141.8, 123.0, 120.4, 115.2, 110.6, 17.8, 15.8; IR (ATR, Diamond): v (cm⁻¹)=3076 (vw), 2236 (m), 1579 (m), 1560 (m), 1435 (vs), 1092 (m). 945 (m), 810 (s), 764 (m), 579 (m), 560 (m).

### Example 8: Preparation of 2-[2-[(3-cyano-2-pyridyl)sulfanyl]ethyl]propanedinitrile

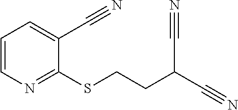 [SMILES: N#Cc1cccnc1SCCC(C#N)C#N]

In a 3 necked 50 mL round bottom flask equipped with magnetic bar, thermometer and nitrogen inlet was charged DMF (10 mL). To this were added 5-amino-2,3-dihydrothiophene-4-carbonitrile (911 mg, 1 equiv, 7.07 mmol, 98 mass %) in one portion and 2-chloropyridine-3-carbonitrile (1.00 g, 7.07 mmol, 98 mass %). The reaction mixture took a light yellowish color. It was then cooled to −10° C. (Ice and brine solution), and sodium tert-butanolate (2.2 equiv., 15.6 mmol) was added in three portions (0.5 g per portion) at −10° C. (5-6° C. Exotherm was observed). The reaction was stirred at −10° C. for one hour, then water was added (10 mL), followed by cold 15 mL 2N aq. HCl and cold 5 mL of ethyl acetate. The mixture had then reached a temperature of 5° C. and a precipitation had occurred upon the addition of the ethyl acetate The solid was filtered through sintered funnel and washed with water (10 mL). The solid was then dried under reduced pressure at 50° C. to deliver 1.55 g of a white solid. Q-NMR gave a reading of 94 mass % and the yield thus obtained is 90%. ¹H NMR (400 MHz, D6-DMSO): δ (ppm)=8.71 (dd, J=4, 1.5 Hz, 1H), 8.26 (dd, J=8, 0.8 Hz, 1H), 7.36 (dd, J=8, 4 Hz, 1H), 4.95 (t, J=6 Hz, 1H), 3.43 (t, J=7 Hz, 2H), 2.44 (q, J=7 Hz, 2H).

### Example 9: Preparation of 2-(1-Cyanocyclopropyl)benzonitrile

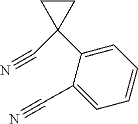 [SMILES: N#Cc1ccccc1C1(C#N)CC1]

To a solution of 5-amino-2,3-dihydrothiophene-4-carbonitrile (127 mg, 94% w/w, 0.95 mmol) and 2-fluorobenzonitrile (1 equiv., 0.95 mmol) in 7.6 mL DMF were added, at room temperature, 2.2 equivalents of a 2M sodium tert-butanolate (2.09 mmol) solution in THF. After overnight stirring at room temperature, the reaction mixture was heated up to 80° C. for 24 hours, before being allowed to cool down to room temperature. It was then poured onto 10 mL of saturated, aqueous NH₄Cl solution and stirred for 10 minutes. The resulting mixture was then acidified with 10 mL of aqueous 1N HCl and extracted three times with 20 mL of ethyl acetate. The combined organic layers were washed with 10 mL aq. sat. NaHCO₃, then with 10 mL of brine (twice), and finally dried over solid MgSO₄ and filtered. The solvent was removed under reduced pressure to deliver 164 mg of the crude desired product as an orange oil. The crude was quantified by Q-NMR to give a strength (w/w) of 65% and therefore the reaction gave a chemical yield of 66%. The crude was purified by chromatography column to give 88 mg of the pure, desired product 2-(1-Cyanocyclopropyl)benzonitrile as a white solid (Isolated Yield 56%) m.p.=106.2-108.1° C.; ¹H NMR (CDCl₆, 400 MHz) δ (ppm)=7.73 (dd, 1H, J=0.91, 7.81 Hz), 7.63 (dt, 1H J=0.91, 7.81 Hz), 7.54 (dd, 1H, J=0.91, 7.81 Hz), 7.50 (dt, 1H J=0.91, 7.81 Hz), 1.82-1.93 (m, 2H), 1.45-1.55 (m, 2H); ¹³C NMR (CDCl₃, 101 MHz) δ 139.1, 133.7, 133.3, 130.9, 129.3, 120.9, 116.5, 114.8, 16.1, 13.1; IR (ATR, Diamond): v (cm⁻¹)=3108 (w), 3072 (w), 2233 (s), 2223 (s), 1599 (m), 1451 (m), 1422 (m), 1051 (m), 946 (m), 773 (vs), 559 (s), 493 (m)

### Example 10: Preparation of 2-[2-(2-cyanophenyl)sulfanylethyl]propanedinitrile

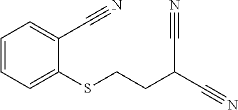 [SMILES: N#Cc1ccccc1SCCC(C#N)C#N]

In a 3 necked 250 mL round bottom flask equipped with magnetic bar, thermometer and nitrogen inlet was charged DMF (58 mL). To this were added 5-amino-2,3-dihydrothiophene-4-carbonitrile (917 mg, 1 equiv., 7.27 mmol) in one portion and 2-chlorobenzonitrile (1.00 g, 7.27 mmol). Sodium tert-butanolate (2.2 equiv., 16 mmol) was added in three portions (˜0.5 g per portion) at room temperature. The reaction was stirred for one hour, then cooled down to 0° C. before water was added (5 mL). A solid precipitation was observed. The mixture was further acidified by dropwise addition of cold 2N aq. HCl and extra 20 mL of water. The solid was filtered through sintered funnel and washed with water (10 mL). The solid was then dried under reduced pressure at 50° C. to deliver 1.06 g of a white solid. Q-NMR gave a reading of 91 mass % and the yield thus obtained is 59%. ¹H NMR (CDCl₃, 400 MHz) δ (ppm)=2.34 (q, J=7 Hz, 2H); 3.26 (t, J=7 Hz, 2H): 4.22 (t, J=6 Hz, 1H); 7.43 (td, J=7.43, 1.53 Hz, 1H) 7.52-7.65 (m, 2H) 7.68-7.75 (m, 1H).

### Example 11: Preparation of 1-pyridazin-3-ylcyclopropanecarbonitrile

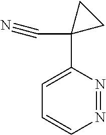 [SMILES: N#CC1(c2cccnn2)CC1]

To a solution of 5-amino-2,3-dihydrothiophene-4-carbonitrile (86 mg, 94% w/w, 0.64 mmol) and 3-bromopyridazine (1 equiv., 0.64 mmol) in 5.1 mL DMF were added, at room temperature, 2.2 equivalents of a 2M sodium tert-butanolate (1.4 mmol) solution in THF. After overnight stirring at room temperature, the reaction mixture was heated up to 60° C. for 24 hours, before being allowed to cool down to room temperature. It was then poured onto 10 mL of saturated, aqueous NH₄Cl solution and stirred for 10 minutes. The resulting mixture was then acidified with 10 mL of aqueous 1N HCl and extracted three times with 20 mL of ethyl acetate. The combined organic layers were washed with 10 mL aq. sat. NaHCO₃, then with 10 mL of brine (twice), and finally dried over solid MgSO₄ and filtered. The initial aqueous phase (NH₄Cl acidified by HCl) was extracted twice with 20 mL of CHCl₃ The organic phases are combined and the solvents removed under reduced pressure to deliver 46 mg of the crude desired product as an orange crystalline solid The crude was quantified by Q-NMR to give a strength (w/w) of 50% and therefore the reaction gave a chemical yield of 42%. The crude was purified by chromatography column to give 36 mg of the pure, desired product 1-pyridazin-3-ylcyclopropanecarbonitrile as white pinkish needles (Isolated Yield 39%).

Alternatively, the title product can be made from 3-(benzenesulfonyl)pyridazine instead of 3-bromopyridazine. Indeed, to a solution of 3-(benzenesulfonyl)pyridazine (101 mg, 0.436 mmol, 95 mass %) and 5-amino-2,3-dihydrothiophene-4-carbonitrile (58 mg, 94% w/w, 0.436 mmol) in DMF (3.4 mL) at room temperature is added a 2M solution of sodium tert-butanolate (2.2 eq.: 0.958 mmol; 480 μL). The reaction mixture is stirred overnight before being heated up to 65° C. for 7 h After cooling down the reaction mixture to room temperature, 10 mL of a saturated aqueous solution of NH₄Cl is added. The resulting mixture is poured into a 50 mL Erlenmeyer and acidified by 10 mL of 1N HCl. It is then extracted 3 times by EtOAc (20 mL each time), the combined organic phases are washed with 10 mL of sat. NaHCO₃, washed further with 10 mL of Brine, then dried over solid MgSO₄ and filtered. The solvents were removed under reduced pressure to deliver 174 mg of the crude desired product as an dark solid. The crude was quantified by Q-NMR to give a strength (w/w) of 52% and therefore the reaction gave a chemical yield of 14%. The crude was purified by chromatography column to give 6 mg of the pure, desired product 1-pyridazin-3-ylcyclopropanecarbonitrile as a white solid (isolated yield is 10%).

Alternatively, the title product can be made from cyclisation of the isolated intermediate 2-(2-pyridazin-3-ylsulfanylethyl)propanedinitrile (example 12) following this protocol. To a solution of 2-2-pyridazin-3-ylsulfanylethyl)propanedinitrile (500 mg, 2.45 mmol) in 5 mL of THF at room temperature were added 1.22 mL of the base sodium tert-butanolate (1 equiv., 2.45 mmol, 2M solution in THF). The reaction mixture was then heated at 65° C. for 2 hours, before being allowed to cool down to room temperature. It was then poured onto 5 mL of a saturated aqueous NH₄Cl solution, stirred for 10 minutes and extracted 3 times with 20 mL of EtOAc. The combined organic layers were dried over solid Na₂SO₄, filtered and the solvent was removed under reduced pressure to deliver 270 mg of the crude desired product as an orange liquid. The crude was quantified by Q-NMR to give a strength (w/w) of 92% and therefore the reaction gave a chemical yield of 70%. The crude was purified by chromatography column to give 240 mg of the pure, desired product 1-pyridazin-3-ylcyclopropanecarbonitrile as a white solid (Purity measured by Q-NMR being 98% (w/w) the isolated Yield is 66%). m.p.=111.7-114.5° C. with gradual degradation up to 115° C.; ¹H NMR (CDCl₃, 400 MHz) δ (ppm)=911 (dd, 1H, J=1.45, 4.72 Hz), 7.91 (dd, 1H, J=1.63, 8.54 Hz), 7.52 (dd, 1H, J=8.54, 4.9 Hz), 2.1-2.2 (m, 2H), 1.83-1.94 (m, 2H); ¹³C NMR (CDCl₃, 101 MHz) δ (ppm) 157.5, 149.9, 126.5, 124.6, 120.9, 21.1, 14.5; IR (ATR, Diamond): v (cm⁻¹)=3100 (w), 3051 (m), 2238 (s), 1579 (m), 1439 (vs), 1136 (s), 948 (s), 845 (s), 798 (s), 732 (s)

### Example 12: Preparation of 2-(2-pyridazin-3-ylsulfanylmethyl)propanedinitrile

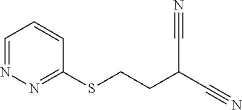 [SMILES: N#CC(C#N)CCSc1cccnn1]

In a 3 necked round bottom flask equipped with magnetic bar, thermometer and nitrogen inlet was charged DMF (10 mL). To this were added 5-amino-2,3-dihydrothiophene-4-carbonitrile (810 mg, 1 equiv., 6.29 mmol, 98 mass %) in one portion and 3-bromopyridazine (1.00 g, 6.29 mmol). Sodium tert-butanolate (2.2 equiv., 13.8 mmol) was added in two portions at room temperature. The reaction was stirred for one hour, then 10 ml of ice-cold water were added and the mixture was acidified with 10 mL of 1N HCl. The aqueous layer was extracted thrice with 50 mL of EtOAc, then the combined organic layers were washed with 20 mL of brine and dried over solid Na₂SO₄. After filtration, the solvents were removed under reduced pressure to deliver 2.66 g of the crude desired product as an orange liquid. The crude was quantified by Q-NMR to give a strength (w/w) of 36.5% and therefore the reaction gave a chemical yield of 70%. The crude was purified by chromatography column to give 912 mg of the pure, desired product 2-(2-pyridazin-3-ylsulfanylethyl)propanedinitrile as a yellow liquid (Purity measured by Q-NMR being 96% (w/w) the isolated Yield is 68%). ¹H NMR (CDCl₃, 400 MHz) δ (ppm)=2.63 (q, J=6.85 Hz, 2H), 3.57 (t, J=6.79 Hz, 2H), 4.13 (t, J=6.79 Hz, 1H), 7.32 (dd, J=8.5, 5 Hz, 1H), 7.37 (dd, J=1.3, 8.5 Hz, 1H), 8.96 (dd, J=1.4, 4.7 Hz, 1H).

### Example 13: Preparation of 1-[5-(trifluoromethyl)-2-pyridyl]cyclopropanecarbonitrile

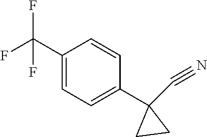 [SMILES: N#CC1(c2ccc(C(F)(F)F)cc2)CC1]

DMF (3.4 mL) was added to 5-amino-2,3-dihydrothiophene-4-carbonitrile (76 mg, 1 equiv., 0.57 mmol, 94 mass %) and 2-chloro-5-(trifluoromethyl)pyridine (106 mg, 0.57 mmol, 97 mass %). Sodium tert-butanolate (2.2 equiv., 0.121 mg, 1.25 mmol) was added in one portion at room temperature. After 30 minutes stirring, the reaction was heated up to 65° C. and stirred for two and a half hours, before being allowed to cool down to room temperature. 10 ml of saturated aqueous NH₄Cl were added and the resulting mixture was stirred for 10 minutes. It was then transferred to a 50 mL Erlenmeyer and acidified with 10 mL of 1N HCl. The aqueous layer was extracted thrice with 20 mL of EtOAc, then the combined organic layers were washed with 20 mL of brine. The combined organic layers were dried over solid Na₂SO₄, filtered and the solvents were removed under reduced pressure to deliver 0.426 g of the crude desired product as an orange liquid. The crude was purified by chromatography column to give 74 mg of the pure, desired product 1-[5-(trifluoromethyl)-2-pyridyl]cyclopropanecarbonitrile as a white powder (isolated Yield is 62%).

Alternatively, the title product can be made from cyclisation of the isolated intermediate 2-[2-[[5-(trifluoromethyl)-2-pyridyl]sulfanyl]ethyl]propanedinitrile (example 14) following this protocol. To a solution of 2-[2-[[5-(trifluoromethyl)-2-pyridyl]sulfanyl]ethyl]propanedinitrile (500 mg, 1.84 mmol) in 5 mL of THF at room temperature were added 0.92 mL of the base sodium tert-butanolate (1 equiv., 1.84 mmol, 2M solution in THF) The reaction mixture was then heated at 65° C. for 3 hours, before being allowed to cool down to room temperature. It was then poured onto 5 mL of a saturated aqueous NH₄Cl solution, stirred for 10 minutes and extracted 3 times with 20 mL of EtOAc The combined organic layers were dried over solid Na₂SO₄, filtered and the solvent was removed under reduced pressure to deliver 506 mg of the crude desired product as an orange liquid. The crude was quantified by Q-NMR to give a strength (w/w) of 68% and therefore the reaction gave a chemical yield of 88%. The crude was purified by chromatography column to give 335 mg of the pure, desired product 1-[5-(trifluoromethyl)-2-pyridyl]cyclopropanecarbonitrile as a white solid (Purity measured by Q-NMR being 96% (w/w) the isolated Yield is 82%). m.p.=74.3-76.5° C., ¹H NMR (CDCl₃, 400 MHz) δ (ppm)=8.72 (dd, 1H, J=0.9, 3.1 Hz), 7.9-8.0 (m. 1H), 7.8-7.9 (m, 1H), 1.9-2.0 (m, 2H), 1.8-1.9 (m, 2H); ¹³C NMR (CDCl₃, 101 MHz) δ 158.7, 146.6, 133.8, 125.0, 121.2, 120.6, 123.4, 20.8, 15.6. ¹⁹F NMR (CDCl₃, 377 MHz) δ −62.33 (s, 3F); IR (ATR, Diamond): v (cm⁻¹)=3062 (vw), 2240 (w), 1606 (m), 1328 (s), 1122 (vs), 1079 (s), 1016 (m)

### Example 14: Preparation of 2-[2-[[5-(trifluoromethyl)-2-pyridyl]sulfanyl]ethyl]propanedinitrile

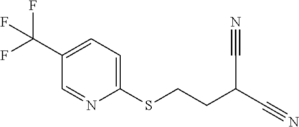 [SMILES: N#CC(C#N)CCSc1ccc(C(F)(F)F)cn1]

In a 3 necked 50 mL round bottom flask equipped with magnetic bar, thermometer and nitrogen inlet was charged DMF (10 mL). To this were added 5-amino-2,3-dihydrothiophene-4-carbonitrile (690 mg, 1 equiv, 5.3 mmol, 98 mass %) in one portion and 2-chloro-5-(trifluoromethyl)pyridine (1.00 g, 5.3 mmol, 97 mass %). It was then cooled to −10° C. (Ice and brine solution), and sodium tert-butanolate (2.2 equiv., 12 mmol) was added in three portions at −10° C. The reaction was stirred at −10° C. for one hour, then ice-cold water was added (5 mL), followed by cold 10 mL 1N aq. HCl. The aqueous layer was then extracted thrice with 20 mL of EtOAc, and the combined organic layers washed with 20 mL of brine. The combined organic layers were dried over solid Na₂SO₄, filtered and the solvents were removed under reduced pressure to deliver 3.3 g of the crude desired product as an orange liquid. The crude was quantified by Q-NMR to give a strength (w/w) of 33% and therefore the reaction gave a chemical yield of 75%. The crude was purified by chromatography column to give 1.058 g of the pure, desired product 2-[2-[[5-(trifluoromethyl)-2-pyridyl]sulfanyl]ethyl]propanedinitrile as a white solid (Purity measured by Q-NMR being 90% (w/w) the isolated Yield is 62%). ¹H NMR (CDCl₃, 400 MHz) δ (ppm)=2.54 (q, J=6.85 Hz, 2H), 3.48 (t, J=6.66 Hz, 2H), 4.03 (t, J=7.34 Hz, 1H), 7.32 (d, J=8.44 Hz, 1H), 7.74 (dd, J=8.44, 2.20 Hz, 1H), 8.71 (s, 1H).

### Example 15: Preparation of 2-(1-cyanocyclopropyl)-5-(trifluoromethyl)benzonitrile

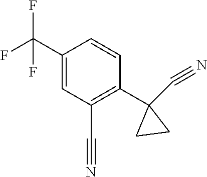 [SMILES: N#Cc1cc(C(F)(F)F)ccc1C1(C#N)CC1]

In a 3 necked round bottom flask equipped with magnetic bar, thermometer and nitrogen inlet was charged DMF (3 mL). To this were added 5-amino-2,3-dihydrothiophene-4-carbonitrile (79 mg, 1 equiv., 0.59 mmol, 94 mass %) and 2-fluoro-5-(trifluoromethyl)benzonitrile (0.111 g, 1 equiv., 0.59 mmol). Sodium tert-butanolate (2.2 equiv., 1.29 mmol) was added at room temperature. The reaction was stirred for three hours, then 10 ml of saturated aqueous NH₄Cl were added and the mixture was acidified with 10 mL of 1N HCl. The aqueous layer was extracted thrice with 20 mL of EtOAc, the combined organic layers were washed with 10 ml of aqueous saturated NaHCO₃, and dried with 20 mL of brine. The combined organic layers were dried over solid Na₂SO₄, filtered and the solvents were removed under reduced pressure to deliver 0.242 g of the crude desired product as a yellow liquid. The crude was quantified by Q-NMR to give a strength (w/w) of 44.7% and therefore the reaction gave a chemical yield of 84%. The crude was purified by chromatography column to give 87 mg of the pure, desired product 2-(1-cyanocyclopropyl)-5-(trifluoromethyl)benzonitrile as a white solid (the isolated yield is 63%).

Alternatively, the title product can be made from cyclisation of the isolated intermediate 2-[2-[2-cyano-4-(trifluoromethyl)phenyl]sulfanylethyl]propanedinitrile (example 16) following this protocol. In a 3 necked round bottom flask equipped with magnetic bar, thermometer and nitrogen inlet was charged THF (4 mL). To this was added 2-[2-[2-cyano-4-(trifluoromethyl)phenyl]sulfanylethyl]propanedinitrile (0.5 g, 1 equiv., 1.69 mmol). Sodium tert-butanolate (1 equiv., 1.69 mmol) was added at room temperature. The reaction was heated up to 65° C. and stirred for three hours, then allowed to cool down to room temperature. 5 ml of saturated aqueous NH₄Cl were added and the aqueous layer was extracted thrice with 20 mL of EtOAc, the combined organic layers were dried over solid Na₂SO₄, filtered and the solvents were removed under reduced pressure to deliver 0.45 g of the crude desired product as an orange liquid. The crude was quantified by Q-NMR to give a strength (w/w) of 62% and therefore the reaction gave a chemical yield of 69%. The crude was purified by chromatography column to give 249 mg of the pure, desired product 2-(1-cyanocyclopropyl)-5-(trifluoromethyl)benzonitrile as a yellow solid (Purity measured by Q-NMR being 93% (w/w) the isolated yield is 58%) m.p.=104.0-105.9° C.; ¹H NMR (CDCl₃, 400 MHz) δ (ppm)=7.99 (d, J=1.45 Hz, 1H), 7.89 (dd, J=8.36, 1.45 Hz, 1H), 7.70 (d, 1H, J=8 Hz), 1.96-1.93 (m, 2H), 1.56-1.52 (m, 2H); ¹³C NMR (CDCl₃, 101 MHz) δ 142.7, 131.7, 132.2, 130.6, 130.1, 120.0, 122.5, 115.8, 115.2, 16.3, 13.2; ¹⁹F NMR (CDCl₃, 377 MHz) δ −63.23 (s, 3F); IR (ATR, Diamond). v (cm⁻¹)=3076 (vw), 2238 (w), 1615 (w), 1327 (s), 1162 (s), 1128 (vs), 1095 (s), 857 (m), 733 (m)

### Example 16: Preparation of 2-[2-[2-cyano-4-(trifluoromethyl)phenyl]sulfanylethyl]propanedinitrile

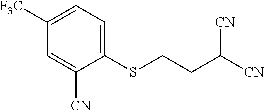 [SMILES: N#Cc1cc(C(F)(F)F)ccc1SCCC(C#N)C#N]

In a 3 necked round bottom flask equipped with magnetic bar, thermometer and nitrogen inlet was charged DMF (10 mL). To this were added 5-amino-2,3-dihydrothiophene-4-carbonitrile (667 mg. 1 equiv., 5.29 mmol) and 2-fluoro-5-(trifluoromethyl)benzonitrile (1 g, 1 equiv., 5.29 mmol). The mixture was cooled down at −40° C., and the base Sodium tert-butanolate (2.2 equiv., 11.6 mmol) was added The reaction was stirred for one hour, then 5 ml of ice cold water were added dropwise (temperature not exceeding −30° C.) followed by 10 ml of 2N HCl. After being allowed to reach room temperature, the aqueous layer was extracted thrice with 20 mL of EtOAc, the combined organic layers were dried over solid Na₂SO₄, filtered and the solvents were removed under reduced pressure to deliver 4.4 g of the crude desired product as a yellow liquid. The crude was quantified by Q-NMR to give a strength (w/w) of 27% and therefore the reaction gave a chemical yield of 77%. The crude was purified by chromatography column to give 1.1 g of the pure, desired product 2-[2-[2-cyano-4-(trifluoromethyl)phenyl]sulfanylethyl]propanedinitrile as an orange solid (Purity measured by Q-NMR being 80% (w/w) the isolated yield is 58%). ¹H NMR (CDCl₃, 400 MHz) δ (ppm)=7.94 (d. J=1.63 Hz, 1H), 7.83 (dd, J=8.51, 1.63 Hz, 1H), 7.60 (d, J=8.38 Hz, 1H), 4.15 (t, J=7.07 Hz, 1H), 3.37 (t, J=7.07 Hz, 2H), 2.42 (q, J=7.07 Hz, 2H). (16% of the cyclized product 2-(1-cyanocyclopropyl)-5-(trifluoromethyl)benzonitrile was also isolated from the column in another fraction).

### Example 17: Preparation of 3-(1-cyanocyclopropyl)-5-(trifluoromethyl)pyridine-2-carbonitrile

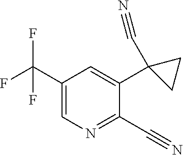 [SMILES: N#Cc1ncc(C(F)(F)F)cc1C1(C#N)CC1]

To 2.8 mL of DMF were added 5-amino-2,3-dihydrothiophene-4-carbonitrile (77 mg, 1.2 equiv., 0.57 mmol, 94 mass %) and 3-chloro-5-(trifluoromethyl)pyridine-2-carbonitrile (100 mg, 0.47 mmol, 1 equiv., 98 mass %). Sodium tert-butanolate (2.2 equiv., 1.04 mmol) was added at room temperature. The reaction was stirred for three hours, then 10 ml of saturated aqueous NH₄Cl were added and the mixture was acidified with 10 mL of 1N HCl. The aqueous layer was extracted thrice with 20 mL of EtOAc, the combined organic layers were washed with 10 ml of aqueous saturated NaHCO₃, and washed with 20 mL of brine. The combined organic layers were dried over solid Na₂SO₄, filtered and the solvents were removed under reduced pressure to deliver 0.178 g of the crude desired product as a brownish liquid. The crude was quantified by Q-NMR to give a strength (w/w) of 48% and therefore the reaction gave a chemical yield of 76%. The crude was purified by chromatography column to give 88 mg of the pure, desired product 3-(1-cyanocyclopropyl)-5-(trifluoromethyl)pyridine-2-carbonitrile as a light yellow oil (95% purity assumed; 74% isolated yield).

Alternatively, the title product can be made from cyclisation of the isolated intermediate 2-[2-[2-cyano-5-(trifluoromethyl)-3-pyridyl]sulfanyl]ethyl]propanedinitrile (example 18) following this protocol. In a 3 necked 25 mL round bottom flask equipped with magnetic bar, thermometer and nitrogen inlet was charged THF (5 mL). To this was added 2-[2-[[2-cyano-5-(trifluoromethyl)-3-pyridyl]sulfanyl]ethyl]-propanedinitrile (0.2 g, 1 equiv., 0.675 mmol). Sodium tert-butanolate (1 equiv., 0.675 mmol) was added at room temperature. The reaction was stirred at this temperature for one hour, then was diluted with 10 mL of water and acidified with 5 mL of aqueous HCl 2N. The aqueous layer was extracted thrice with 50 mL of EtOAc, the combined organic layers were dried over solid Na₂SO₄, filtered and the solvents were removed under reduced pressure to deliver 0.21 g of the crude desired product. The crude was quantified by Q-NMR to give a strength (w/w) of 49% and therefore the reaction gave a chemical yield of 64%. The crude was purified by chromatography column to give 106 mg of the pure, desired product 3-(1-cyanocyclopropyl)-5-(trifluoromethyl)pyridine-2-carbonitrile as a yellow liquid (Purity measured by Q-NMR being 92% (w/w) the isolated yield is 61%). m.p.=101.5-103.6° C.; ¹H NMR (CDCl₃, 400 MHz) δ (ppm)=8.97-8.95 (m, 1H), 8.12-8.09 (m, 1H), 2.09-1.95 (m, 2H), 1.65-1.54 (m, 2H); ¹³C NMR (CDCl₃, 101 MHz) δ (ppm)=147.6, 138.9, 136.6, 136.0, 129.6, 119.3, 122.0, 114.2, 16.5, 11.6; ¹⁹F NMR (CDCl₃, 377 MHz) δ −62.78 (s, 3F), IR (ATR, Diamond): v (cm⁻¹)=3074 (vw), 2244 (w), 1566 (w), 1421 (m), 1346 (s), 1289 (vs), 1164 (s), 1147 (s), 1133 (s), 1085 (s), 933 (m)

### Example 18: Preparation of 2-[2-[[2-cyano-5-(trifluoromethyl)-3-pyridyl]sulfanyl]ethyl]propanedinitrile

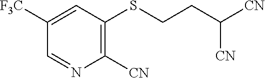 [SMILES: N#Cc1ncc(C(F)(F)F)cc1SCCC(C#N)C#N]

In a 3 necked round bottom flask equipped with magnetic bar, thermometer and nitrogen inlet was charged DMF (10 mL). To this were added 5-amino-2,3-dihydrothiophene-4-carbonitrile (623 mg, 1 equiv., 4.84 mmol, 98 mass %) and 3-chloro-5-(trifluoromethyl)pyridine-2-carbonitrile (1 g, 4.84 mmol) The mixture was cooled down at −40° C., and the base Sodium tert-butanolate (2.2 equiv., 10.7 mmol) was added. The reaction was stirred for half an hour, then 10 ml of ice cold water were added dropwise (temperature not exceeding −30° C.) followed by 10 ml of 2N HCl. After being allowed to reach room temperature, the aqueous layer was extracted thrice with 50 mL of EtOAc, the combined organic layers were washed with 20 mL of brine, dried over solid Na₂SO₄, filtered and the solvents were removed under reduced pressure to deliver 2.9 g of the crude desired product as an orange liquid. The crude was quantified by Q-NMR to give a strength (w/w) of 35% and therefore the reaction gave a chemical yield of 70%. The crude was purified by chromatography column to give 0.92 g of the pure, desired product 2-[2-[[2-cyano-5-(trifluoromethyl)-3-pyridyl]sulfanyl]ethyl]propanedinitrile as an orange solid (Purity measured by Q-NMR being 93% (w/w) the isolated yield is 60%) ¹H NMR (D6-DMSO, 400 MHz) δ (ppm)=9-8.93 (m, 1H), 8.55-8.49 (m, 1H), 4.89 (t, J=7.1 Hz, 1H), 3.46 (t, J=7.1 Hz, 2H), 2.39 (q, J=7.1 Hz, 2H)

### Example 19: Preparation of 2-(1-cyanocyclopropyl)-4-(trifluoromethyl)benzonitrile

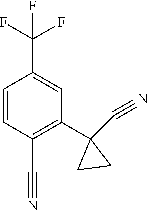 [SMILES: N#Cc1ccc(C(F)(F)F)cc1C1(C#N)CC1]

In a 20 mL vial was charged DMF (4.3 mL). To this were added 5-amino-2,3-dihydrothiophene-4-carbonitrile (73 mg, 1 equiv., 0.54 mmol, 94 mass %) and 2-fluoro-4-(trifluoromethyl)benzonitrile (0.103 g, 1 equiv., 0.54 mmol). Sodium tert-butanolate (2.2 equiv, 1.2 mmol) was added at room temperature The reaction was stirred for six hours, then 10 ml of saturated aqueous NH₄Cl were added. After transfer to an Erlenmeyer the mixture was acidified with 10 mL of 1N HCl. The aqueous layer was extracted thrice with 20 mL of EtOAc, the combined organic layers were washed with 10 ml of aqueous saturated NaHCO₃, and washed with 20 mL of brine. The combined organic layers were dried over solid Na₂SO₄, filtered and the solvents were removed under reduced pressure to deliver 0.15 g of the crude desired product as an orange solid. The crude was quantified by Q-NMR to give a strength (w/w) of 67% and therefore the reaction gave a chemical yield of 78%. The crude was purified by chromatography column to give 89 mg of the pure, desired product 2-(1-cyanocyclopropyl)-4-(trifluoromethyl)benzonitrile as a white solid (the isolated yield is 69%).

m.p.=94.8-97.7° C.; ¹H NMR (CDCl₃, 400 MHz) δ (ppm)=7.89 (d, 1H, J=7.6 Hz), 7.7-7.8 (m, 2H), 1.9-2.0 (m, 2H), 1.5-1.6 (m, 2H); ¹³C NMR (CDCl₃, 101 MHz) δ (ppm)=140.3, 135.1, 134.4, 127.9, 126.3, 120.1, 122.6, 118.4, 115.2, 16.3, 13.2, ¹⁹F NMR (₁-CDCl₃, 377 MHz) δ (ppm)=−63.46 (s, 3F); IR (ATR, Diamond): v (cm⁻¹)=3093 (vw), 2925 (vw), 2238 (w). 1428 (w), 1338 (s), 1295 (s). 1280 (s), 1176 (s), 1130 (vs), 1084 (s), 952 (m). 844 (m), 564 (m)

### Example 20: Preparation of 2-[2-[2-cyano-5-(trifluoromethyl)phenyl]sulfanylethyl]propanedinitrile

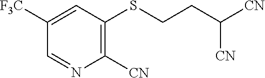 [SMILES: N#Cc1ncc(C(F)(F)F)cc1SCCC(C#N)C#N]

In a 20 mL vial was charged DMF (4.4 mL). To this were added 5-amino-2,3-dihydrothiophene-4-carbonitrile (75 mg, 1 equiv., 0.56 mmol, 94 mass %) and 2-fluoro-4-(trifluoromethyl)benzonitrile (0.105 g, 1 equiv., 0.56 mmol). Sodium tert-butanolate (2.2 equiv., 1.22 mmol) was added at room temperature. The reaction was stirred for ten minutes, then 10 ml of saturated aqueous NH₄Cl were added. After transfer to an Erlenmeyer the mixture was acidified with 10 mL of 1N HCl. The aqueous layer was extracted thrice with 20 mL of EtOAc, the combined organic layers were washed with 10 mL of brine. The combined organic layers were dried over solid Na₂SO₄, filtered and the solvents were removed under reduced pressure to deliver 0.379 g of the crude desired product as an orange liquid. The crude was purified by chromatography column to give 112 mg of the pure, desired product 2-[2-[2-cyano-5-(trifluoromethyl)phenyl]sulfanylethyl]propanedinitrile as a white solid (the isolated yield is 68%). m.p.=110.7-113.2° C. ¹H NMR (400 MHz, CDCl₃) δ (ppm)=7.87 (1H, d, J=7.99 Hz), 7.78 (1H, s), 7.68 (1H, dd, J=7.99, 0.73 Hz), 4.19 (1H, t, J=7.27 Hz), 3.36 (2H, t, J=6.90 Hz), 2.42 (2H, q, J=7.14 Hz) ¹³C NMR (₁-CDCl₃, 101 MHz) δ 139.9, 135.3, 134.6, 127.6, 124.8, 118.6, 118.5, 123.2, 115.7, 111.6, 30.8, 30.1, 213; IR (ATR, Diamond): v (cm⁻¹)=2916 (vw), 2230 (vw), 1321 (vs), 1176 (s), 1132 (s), 1083 (m), 845 (m)

### Example 21: Preparation of 1-(6-chloropyridazin-3-yl)cyclopropanecarbonitrile

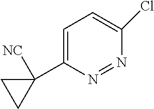 [SMILES: N#CC1(c2ccc(Cl)nn2)CC1]

In a 20 mL vial was charged DMF (3.2 mL). To this were added 5-amino-2,3-dihydrothiophene-4-carbonitrile (100 mg, 1 equiv., 0.79 mmol) and 3,6-dichloropyridazine (0.118 g, 1 equiv., 0.79 mmol) Sodium tert-butanolate (2 equiv., 1.58 mmol) was added at room temperature. The reaction was heated to 65° C. and stirred for six hours, before being allowed to cool down to room temperature. 10 ml of saturated aqueous NH₄Cl were added. After transfer to an Erlenmeyer the mixture was acidified with 10 mL of 1N HCl. The aqueous layer was extracted thrice with 30 mL of DCM, the combined organic layers were dried over solid Na₂SO₄, filtered and the solvents were removed under reduced pressure to deliver 0.244 g of the crude desired product. The crude was quantified by Q-NMR to give a strength (w/w) of 22% and therefore the reaction gave a chemical yield of 37%. The crude was purified by chromatography column to give 33 mg of the pure, desired product 1-(6-chloropyridazin-3-yl)cyclopropanecarbonitrile (the isolated yield is 23%). (15 mg (9% yield) of [6-(1-cyanocyclopropyl)pyridazin-3-yl]thiocyanate were also isolated in another fraction).

Alternatively, the title product can be made from cyclisation of the isolated intermediate 2-[2-(6-chloropyridazin-3-yl)sulfanylethyl]propanedinitrile (example 22) following this protocol.

In a 3 necked Round bottom flask equipped with magnetic bar, thermometer and nitrogen inlet was charged THF (5 mL). To this was added 2-[2-(6-chloropyridazin-3-yl)sulfanylethyl]propanedinitrile (0.5 g, 1 equiv., 2.09 mmol) Sodium tert-butanolate (1 equiv., 2.09 mmol) was added at room temperature. The reaction was heated up to 65° C. and stirred for three hours, then allowed to cool down to room temperature. 5 ml of water were added then acidified with 10 mL of 2N HCl the aqueous layer was extracted thrice with 20 mL of EtOAc, the combined organic layers were dried over solid Na₂SO₄, filtered and the solvents were removed under reduced pressure to deliver 0.36 g of the crude desired product as an orange liquid. The crude was purified by chromatography column to give 202 mg of the pure, desired product 1-(6-chloropyridazin-3-yl)cyclopropanecarbonitrile as a white solid (Purity measured by Q-NMR being 97% (w/w) the isolated yield is 50%). m.p.=134.2-136.6° C.; ¹H NMR (CDCl₃, 400 MHz) δ (ppm)=7.88 (d, 1H, J=8.9 Hz), 7.54 (d, 1H, J=9.17 Hz), 2.18-2.08 (m, 2H), 1.99-1.88 (m, 2H); ¹³C NMR (CDCl₃, 101 MHz) δ (ppm)=156.8, 155.7, 128.3, 127.0, 120.5, 21.4, 14.1; IR (ATR, Diamond): v (cm⁻¹)=3046 (m), 2244 (m), 1422 (vs), 1163 (m), 1133 (s), 871 (m)

### Example 22: Preparation of 2-[2-(6-chloropyridazin-3-yl)sulfanylethyl]propanedinitrile

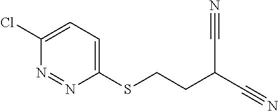 [SMILES: N#CC(C#N)CCSc1ccc(Cl)nn1]

In a 3 necked round bottom flask equipped with magnetic bar, thermometer and nitrogen inlet was charged DMF (10 mL). To this were added 5-amino-2,3-dihydrothiophene-4-carbonitrile (860 mg, 1 equiv., 6.7 mmol, 98 mass %) and 3,6-dichloropyridazine (1 g, 6.7 mmol). The mixture was cooled down at −40° C., and the base Sodium tert-butanolate (2.2 equiv., 15 mmol) was added. The reaction was stirred for 15 minutes, then 10 ml of ice cold water were added. A solid precipitated out, the reaction was further acidified by 2 ml of 2N HCl. The solid precipitate was filtered off on Buchner funnel under vacuum. The white solid obtained was dried under reduced pressure at 50° C. 0.596 g of the pure, desired product 2-[2-(6-chloropyridazin-3-yl)sulfanylethyl]propanedinitrile (Purity measured by Q-NMR being 98% (w/w) the isolated yield is 36%). ¹H NMR (CDCl₃, 400 MHz) δ (ppm)=7.38-7.31 (m, 2H), 4.07 (t, J=6.9 Hz, 1H), 3.55 (t, J=6.55 Hz, 2H), 2.63 (q, J=6.92 Hz, 2H).

### Example 23: Preparation of 1-[2-(trifluoromethyl)-4-pyridyl]cyclopropanecarbonitrile

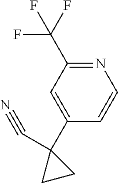 [SMILES: N#CC1(c2ccnc(C(F)(F)F)c2)CC1]

In a 20 mL vial was charged DMF (3.2 mL). To this were added 5-amino-2,3-dihydrothiophene-4-carbonitrile (72 mg, 1 equiv., 0.534 mmol, 94 mass %) and 4-chloro-2-(trifluoromethyl)pyridine (0.1 g, 1 equiv., 0.534 mmol, 97 mass %). Sodium tert-butanolate (2.4 equiv., 1.28 mmol) was added at room temperature. The reaction was stirred one hour at this temperature, and then was heated to 65° C. and stirred for three hours, before being allowed to cool down to room temperature. 10 ml of saturated aqueous NH₄Cl were added. After transfer to an Erlenmeyer the mixture was acidified with 10 mL of 1N HCl. The aqueous layer was extracted thrice with 25 mL of EtOAc, the combined organic layers were dried over solid Na₂SO₄, filtered and the solvents were removed under reduced pressure to deliver 0.383 g of the crude desired product as an orange oil. The crude was quantified by Q-NMR to give a strength (w/w) of 23% and therefore the reaction gave a chemical yield of 78%. The crude was purified by chromatography column to give 75 mg of the pure, desired product 1-[2-(trifluoromethyl)-4-pyridyl]cyclopropanecarbonitrile (97 mass %, isolated yield is 65%).

Alternatively, the title product can be made from cyclisation of the isolated intermediate 2-[2-[[2-(trifluoromethyl)-4-pyridyl]sulfanyl]ethyl]propanedinitrile (example 24) following this protocol.

In a 3 necked round bottom flask equipped with magnetic bar, thermometer and nitrogen inlet was charged THF (5 mL). To this was added 2-[2-[[2-(trifluoromethyl)-4-pyridyl]sulfanyl]ethyl]propanedinitrile (0.5 g, 1 equiv., 1.84 mmol). Sodium tert-butanolate (1 equiv., 1.84 mmol) was added at room temperature. The reaction was heated up to 65° C. and stirred for three hours, then allowed to cool down to room temperature. The reaction mixture was then poured onto 5 ml of aqueous saturated NH₄Cl, stirred for 10 minutes and the aqueous layer was extracted thrice with 20 mL of EtOAc, the combined organic layers were dried over solid Na₂SO₄, filtered and the solvents were removed under reduced pressure to deliver 0.45 g of the crude desired product as an orange liquid. The crude was quantified by Q-NMR to give a strength (w/w) of 55% and therefore the reaction gave a chemical yield of 63%. The crude was purified by chromatography column to give 0.2 g of the pure, desired product 1-[2-(trifluoromethyl)-4-pyridyl]cyclopropanecarbonitrile as a white solid (Purity measured by Q-NMR being 96% (w/w) the isolated yield is 52%). m.p.=58.8-61.2° C.; ¹H NMR (CDCl₃, 400 MHz) δ (ppm)=8.71 (d, 1H, J=5.1 Hz), 7.49 (d, 1H, J=1.5 Hz), 7.42 (dd, 1H, J=1.8, 5.1 Hz), 2.02-1.95 (m, 2H), 1.63-1.58 (m, 2H); ¹³C NMR (CDCl₃, 101 MHz) δ (ppm)=150.5, 149.2, 147.9, 122.4, 120.1, 121.3, 116.1, 20.5, 13.8; ¹⁹1F NMR (CDCl₃, 377 MHz) δ (ppm)=−68.12 (s, 3F); IR (ATR, Diamond): v (cm⁻¹)=3026 (vw), 2926 (vw), 2242 (w), 1610 (m), 1429 (m), 1340 (m), 1310 (m), 1183 (s), 1128 (vs), 1090 (s), 956 (m), 892 (m), 700 (s)

### Example 24: Preparation of 2-[2-[[2-(trifluoromethyl)-4-pyridyl]sulfanyl]ethyl]propanedinitrile

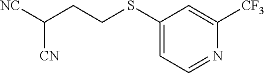 [SMILES: N#CC(C#N)CCSc1ccnc(C(F)(F)F)c1]

In a 3 necked round bottom flask equipped with magnetic bar, thermometer and nitrogen inlet was charged DMF (10 mL). To this were added 5-amino-2,3-dihydrothiophene-4-carbonitrile (709 mg, 1 equiv., 5.51 mmol, 98 mass %) and 4-chloro-2-(trifluoromethyl)pyridine (1 g, 1 equiv., 5.51 mmol). The mixture was cooled at −10° C., and Sodium tert-butanolate (2.2 equiv., 12.1 mmol) was added at this temperature. The reaction was stirred for thirty minutes, then 5 ml of ice cold water were added, followed by ice cold 10 ml of 1N HCl. The aqueous layer was extracted thrice with 20 mL of EtOAc, the combined organic layers were washed with 20 mL of brine. The combined organic layers were dried over solid Na₂SO₄, filtered and the solvents were removed under reduced pressure to deliver 3.5 g of the crude desired product as an orange liquid. The crude was quantified by Q-NMR to give a strength (w/w) of 38% and therefore the reaction gave a chemical yield of 88%. The crude was purified by chromatography column to give 1.02 g of the pure, desired product 2-[2-[[2-(trifluoromethyl)-4-pyridyl]sulfanyl]ethyl]propanedinitrile as a white solid (Purity measured by Q-NMR being 91% (w/w) the isolated yield is 62%). ¹H NMR (CDCl₃, 400 MHz) δ (ppm)=2.46 (q, J=7.09 Hz, 2H), 3.35 (t, J=7.07 Hz, 2H), 4.06 (t, J=6.94 Hz, 1H), 7.32 (d, J=5.25 Hz, 1H), 7.52 (s, 1H), 8.60 (d, J=5.25 Hz, 1H)

### Example 25: Preparation of 1-(3-phenylisoxazol-5-yl)cyclopropanecarbonitrile

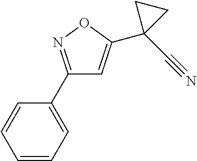 [SMILES: N#CC1(c2cc(-c3ccccc3)no2)CC1]

In a 20 mL vial was charged DMF (4.4 mL). To this were added 5-amino-2,3-dihydrothiophene-4-carbonitrile (75 mg, 1 equiv., 0.557 mmol, 94 mass %) and 5-chloro-3-phenyl-isoxazole (0.1 g, 1 equiv., 0.557 mmol). Sodium tert-butanolate in THF (2M) (22 equiv., 1.28 mmol) was added at room temperature The reaction was stirred overnight at this temperature, and then was heated to 65° C. and stirred for 24 hours, before being allowed to cool down to room temperature. 10 ml of saturated aqueous NH₄Cl were added. After transfer to an Erlenmeyer the mixture was acidified with 10 mL of 1N HCl. The aqueous layer was extracted thrice with 20 mL of EtOAc, the combined organic layers were dried over solid Na₂SO₄, filtered and the solvents were removed under reduced pressure to deliver 0.132 g of the crude desired product as an orange oil. The crude was quantified by Q-NMR to give a strength (w/w) of 21% and therefore the reaction gave a chemical yield of 24%. The crude was purified by chromatography column to give 23 mg of the pure, desired product 1-(3-phenylisoxazol-5-yl)cyclopropanecarbonitrile (Isolated yield is 20%). m.p.=102.2-105.4° C.; ¹H NMR (CDCl₃, 400 MHz) δ (ppm)=7.82-7.78 (m, 2H), 7.50-7.46 (m, 3H), 6.72 (s, 1H), 1.86-1.84 (m, 4H); ¹³C NMR (CDCl₃, 101 MHz) δ (ppm)=166.6, 162.9, 130.3, 128.9, 128.2, 126.7, 119.4, 100.3, 18.2, 8.8; IR (ATR, Diamond): v (cm⁻¹)=3128 (vw), 3026 (vw), 2245 (w), 1603 (m), 1581 (m), 1407 (s), 998 (m), 765 (vs), 692 (s)

### Example 26: Preparation of 1-(5-methylpyrazin-2-yl)cyclopropanecarbonitrile

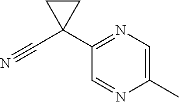

In a 20 mL vial was charged DMF (4.6 mL). To this were added 5-amino-2,3-dihydrothiophene-4-carbonitrile (103 mg, 1 equiv., 0.766 mmol, 94 mass %) and 2-chloro-5-methyl-pyrazine (0.098 g, 1 equiv., 0.766 mmol). Sodium tert-butanolate in THF (2M) (2.2 equiv., 1.69 mmol) was added at room temperature. The reaction was stirred overnight at this temperature, and then was heated to 65° C. and stirred for 24 hours, before being allowed to cool down to room temperature. 10 ml of saturated aqueous NH₄Cl were added. After transfer to an Erlenmeyer the mixture was acidified with 10 mL of 1N HCl. The aqueous layer was extracted thrice with 20 mL of EtOAc, the combined organic layers were dried over solid Na₂SO₄, filtered and the solvents were removed under reduced pressure to deliver 0.105 g of the crude desired product as a brown oil. The crude was purified by chromatography column to give 5 mg of the pure, desired product 1-(5-methylpyrazin-2-yl)cyclopropanecarbonitrile (Isolated yield is 4%). m.p.=57.7-59.2° C.: ¹H NMR (CDCl₃, 400 MHz) δ (ppm)=8.85 (d, 1H, J=1.5 Hz), 8.29 (d, 1H, J=1.1 Hz), 2.56 (s, 3H), 1.82-1.78 (m, 4H); ¹³C NMR (CDCl₃, 101 MHz) δ (ppm)=152.3, 147.3, 143.7, 141.1, 121.0, 21.1, 19.7, 13.0; IR (ATR, Diamond): v (cm⁻¹)=3012 (w), 2240 (m), 1488 (vs), 1337 (s), 1033 (vs)

### Example 27: Preparation of 5-(1-cyanocyclopropyl)-1-phenyl-pyrazole-4-carbonitrile

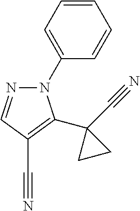 [SMILES: N#Cc1cnn(-c2ccccc2)c1C1(C#N)CC1]

In a 20 mL vial was charged DMF (3.8 mL). To this were added 5-amino-2,3-dihydrothiophene-4-carbonitrile (64 mg, 1 equiv., 0.476 mmol, 94 mass %) and 5-chloro-1-phenyl-pyrazole-4-carbonitrile (0.102 g, 1 equiv., 0.476 mmol). Sodium tert-butanolate in THF (2M) (2.2 equiv., 1.05 mmol) was added at room temperature. The reaction was stirred overnight at this temperature, and then was heated to 65° C. and stirred for 24 hours, before being allowed to cool down to room temperature. 10 ml of saturated aqueous NH₄Cl were added. After transfer to an Erlenmeyer the mixture was acidified with 10 mL of 1N HCl The aqueous layer was extracted thrice with 20 mL of EtOAc, the combined organic layers were dried over solid Na₂SO₄, filtered and the solvents were removed under reduced pressure to deliver 0.088 g of the crude desired product. The crude was quantified by Q-NMR to give a strength (w/w) of 5% and therefore the reaction gave a chemical yield of 4%. m.p.=128.2-129.3° C.; ¹H NMR (CDCl₆, 400 MHz) δ (ppm)=7.94 (s, 1H), 7.65-7.55 (m, 5H), 1.7-1.8 (m, 2H), 1.3-1.4 (m, 2H); ¹³C NMR (CDCl₃, 101 MHz) δ (ppm)=142.4, 141.9, 137.7, 130.0, 129.8, 125.3, 119.5, 112.0, 96.1, 17.3, 5.1; IR (ATR, Diamond): v (cm⁻¹)=3112 (vw), 2236 (s), 1501 (vs), 1405 (m), 767 (s), 695 (m), 662 (m)

### Example 28: Preparation of 1-[6-(trifluoromethyl)-2-pyridyl]cyclopropanecarbonitrile

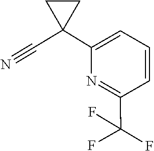 [SMILES: N#CC1(c2cccc(C(F)(F)F)n2)CC1]

In a 20 mL vial was charged DMF (4.9 mL) To this were added 5-amino-2,3-dihydrothiophene-4-carbonitrile (82 mg, 1 equiv., 0.612 mmol, 94 mass %) and 2-fluoro-6-(trifluoromethyl)pyridine (0.102 g, 1 equiv., 0.612 mmol). A solution of Lithium bis(trimethylsilyl)amide (LiHMDS) 1N in THF (2.2 equiv., 1.35 mmol) was added at room temperature. The reaction was stirred overnight at this temperature, and then was heated to 65° C. and stirred for 24 hours, before being allowed to cool down to room temperature. 10 ml of saturated aqueous NH₄Cl were added. After transfer to an Erlenmeyer the mixture was acidified with 10 mL of 1N HCl. The aqueous layer was extracted thrice with 20 mL of EtOAc, the combined organic layers were dried over solid Na₂SO₄, filtered and the solvents were removed under reduced pressure to deliver 0.146 g of the crude desired product 1-[6-(trifluoromethyl)-2-pyridyl]cyclopropanecarbonitrile. The crude was quantified by Q-NMR to give a strength (w/w) of 32% and therefore the reaction gave a chemical yield of 36%. Alternatively, the compound could be made from 2-chloro-6-(trifluoromethyl)pyridine following this protocol: In a 20 mL vial was charged DMF (5.6 mL). To this were added 5-amino-2,3-dihydrothiophene-4-carbonitrile (75 mg, 1 equiv., 0.562 mmol, 94 mass %) and 2-chloro-6-(trifluoromethyl)pyridine (0.102 g, 1 equiv., 0.561 mmol). Sodium tert-butanolate in THF (2M) (2.2 equiv, 1.24 mmol) was added at room temperature. The reaction was stirred one hour at this temperature, and then was heated to 65° C. and stirred for 24 hours, before being allowed to cool down to room temperature. 10 ml of saturated aqueous NH₄Cl were added. After transfer to an Erlenmeyer the mixture was acidified with 10 mL of 1N HCl The aqueous layer was extracted thrice with 20 mL of EtOAc, the combined organic layers were dried over solid Na₂SO₄, filtered and the solvents were removed under reduced pressure to deliver 0.270 g of the crude desired product 1-[6-(trifluoromethyl)-2-pyridyl]cyclopropanecarbonitrile. The crude was quantified by Q-NMR to give a strength (w/w) of 16% and therefore the reaction gave a chemical yield of 35%. The crude was purified by chromatography column to give 25 mg of the pure, desired product 1-[6-(trifluoromethyl)-2-pyridyl]cyclopropanecarbonitrile as a white solid (the isolated yield is 20%)

Alternatively, the title product can be made from cyclisation of the isolated intermediate 2-[2-[[6-(trifluoromethyl)-2-pyridyl]sulfanyl]ethyl]propanedinitrile (example 29) following this protocol.

In a 20 mL vial was charged DMF (1.16 mL). To this was added 2-[2-[[6-(trifluoromethyl)-2-pyridyl]sulfanyl]ethyl]propanedinitrile (52 mg, 1 equiv., 0.194 mmol). A solution of Lithium bis(trimethylsilyl)amide (LiHMDS) 1N in THF (1.1 equiv., 0.213 mmol) was added at room temperature. The reaction was heated to 60° C. and stirred for 3 days, before being allowed to cool down to room temperature. 5 ml of saturated aqueous NH₄Cl were added. After transfer to an Erlenmeyer the mixture was acidified with 5 mL of 1N HCl. The aqueous layer was extracted thrice with 10 mL of EtOAc, the combined organic layers were washed with 5 mL of aqueous saturated NaHCO₃ and twice with 10 mL of brine, before being dried over solid Na₂SO₄ and filtered. The solvents were removed under reduced pressure to deliver 0.034 g of the crude desired product 1-[6-(trifluoromethyl)-2-pyridyl]cyclopropanecarbonitrile. The crude was quantified by Q-NMR to give a strength (w/w) of 49% and therefore the reaction gave a chemical yield of 40%. The crude was purified by chromatography column to give 13 mg of the pure, desired product 1-[6-(trifluoromethyl)-2-pyridyl]cyclopropanecarbonitrile as white needles (the isolated yield is 27%). m.p.=75.4-77.3° C.; ¹H NMR (CDCl₃, 400 MHz) δ (ppm)=7.96 (dd, 1H, J=0.92, 7.52 Hz), 7.9 (t, J=7.52 Hz, 1H), 7.57 (dd, 1H, J=0.92, 7.52 Hz), 1.98-1.87 (m, 2H), 1.86-1.76 (m, 2H); ¹³C NMR (CDCl₃, 101 MHz) δ (ppm)=155.7, 148.2, 138.1, 123.7, 121.3, 118.5, 121.1, 20.7, 15.2, ¹⁹F NMR (CDCl₃, 377 MHz) δ (ppm)=−68.44 (s, 3F); IR (ATR, Diamond): v (cm⁻¹)=3084 (vw), 3022 (vw), 2241 (w), 1596 (m), 1461 (m), 1346 (s), 1304 (s), 1277 (mi), 1194 (s), 1148 (vs), 1087 (s), 807 (s), 704 (s)

### Example 29: Preparation of 2-[2-[[6-(trifluoromethyl)-2-pyridyl]sulfanyl]ethyl]propanedinitrile

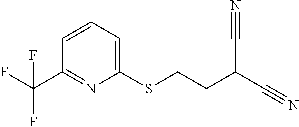 [SMILES: N#CC(C#N)CCSc1cccc(C(F)(F)F)n1]

In a 100 mL RBF was charged DMF (30 mL). To this were added 5-amino-2,3-dihydrothiophene-4-carbonitrile (808 mg, 1 equiv., 6.016 mmol, 94 mass %) and 2-fluoro-6-(trifluoromethyl)pyridine (1 g, 1 equiv., 6.016 mmol, 99 mass %). Sodium tert-butanolate in THF (2M) (2.2 equiv., 13.24 mmol) was added at room temperature. The reaction was stirred one hour at this temperature. 30 ml of saturated aqueous NH₄Cl were added. After transfer to an Erlenmeyer the mixture was acidified with 30 mL of 1N HCl. The aqueous layer was extracted thrice with 50 mL of EtOAc, the combined organic layers were washed with 30 mL of aqueous saturated NaHCO₃ and twice with 30 mL of brine, before being dried over solid Na₂SO₄ and filtered. The solvents were removed under reduced pressure to deliver 1.845 g of the crude desired product as an orange oil. The crude was purified by chromatography column to give 1.41 g of the pure, desired product 2-[2-[[6-(trifluoromethyl)-2-pyridyl]sulfanyl]ethyl]propanedinitrile as an colorless oil which solidified overnight (Isolated yield is 86%) m.p.=61.7-64.9° C.; ¹H NMR (CDCl₃, 400 MHz) δ (ppm)=7.72 (1H, t, J=7.81 Hz), 7.45 (1H, d, J=7.27 Hz), 7.40 (1H, d, J=7.99 Hz), 4.09 (1H, t, J=7.45 Hz), 3.46 (2H, t, J=6.54 Hz), 2.57 (2H, q, J=6.66 Hz); ¹³C NMR (CDCl₃, 101 MHz) δ (ppm)=158.3, 148.2, 137.5, 125.3, 121.2, 116.6, 112.2, 30.7, 26.6, 21.4; IR (ATR, Diamond): v (cm⁻¹)=2914 (vw), 2259 (vw), 1445 (m), 1337 (vs), 1189 (s), 1136 (vs), 1109 (vs), 804 (m), 716 (m)

### Example 30: Preparation of 1-[6-chloro-3-(4-chlorophenyl)pyridazin-4-yl]cyclopropanecarbonitrile

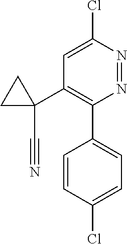 [SMILES: N#CC1(c2cc(Cl)nnc2-c2ccc(Cl)cc2)CC1]

In a 20 mL vial was charged DMF (3 mL). To this were added 5-amino-2,3-dihydrothiophene-4-carbonitrile (49 mg, 1 equiv., 0.368 mmol, 94 mass %) and 4,6-dichloro-3-(4-chlorophenyl)pyridazine (100 mg, 1 equiv., 0.368 mmol, 95 mass %). A solution of Sodium bis(trimethylsilyl)amide (NaHMDS) 1N in THF (2.1 equiv., 0.77 mmol) was added at room temperature. The reaction was stirred for 3 hours. 10 ml of saturated aqueous NH₄Cl were added. After transfer to an Erlenmeyer the mixture was acidified with 10 mL of 1N HCl. The aqueous layer was extracted thrice with 20 mL of EtOAc, the combined organic layers were washed with 10 mL of aqueous saturated NaHCO₃ and twice with 15 mL of brine, before being dried over solid Na₂SO₄ and filtered. The solvents were removed under reduced pressure to deliver 0.131 g of the crude desired product 1-[6-chloro-3-(4-chlorophenyl)pyridazin-4-yl]cyclopropanecarbonitrile as an orange liquid. The crude was purified by chromatography column to give 75 mg of the pure, desired product 1-[6-chloro-3-4-chlorophenyl)pyridazin-4-yl]cyclopropanecarbonitrile as an orange liquid (the isolated yield is 70%). m.p.=152.8-155.5° C. with gradual degradation up to 156° C.; ¹H NMR (CDCl₃, 400 MHz) δ (ppm)=7.74-7.7 (m, 2H), 7.63 (s, 1H), 7.61-7.57 (m, 2H), 1.76-1.65 (m, 2H), 1.26-1.15 (m, 2H); ¹³C NMR (CDCl₃, 101 MHz) δ (ppm)=160.5, 155.9, 136.6, 136.2, 133.3, 130.7, 129.8, 129.2, 120.5, 17.6, 12.4; IR (ATR, Diamond): v (cm⁻¹)=3060 (vw), 2924 (w), 2239 (w), 1393 (vs), 1344 (s), 1093 (vs), 836 (s), 734 (vs)

### Example 31: Preparation of 1-(4-nitrophenyl)cyclopropanecarbonitrile

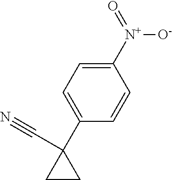 [SMILES: N#CC1(c2ccc([N+](=O)[O-])cc2)CC1]

In a 20 mL vial was charged DMF (3.2 mL). To this were added 5-amino-2,3-dihydrothiophene-4-carbonitrile (100 mg, 1 equiv., 0.792 mmol) and 1-fluoro-4-nitro-benzene (112 mg, 1 equiv., 0.792 mmol). Sodium tert-butanolate (2 equiv., 1.58 mmol) was added at room temperature The reaction was stirred for 3 hours at this temperature, then heated at 65° C. for three extra hours. The reaction was then allowed to cool down to room temperature, then 10 ml of saturated aqueous NH₄Cl were added. After transfer to an Erlenmeyer the mixture was acidified with 10 mL of 1N HCl. The aqueous layer was extracted thrice with 20 mL of DCM, the combined organic layers were dried over solid Na₂SO₄ and filtered The solvents were removed under reduced pressure to deliver 0.294 g of the crude desired product 1-(4-nitrophenyl)cyclopropanecarbonitrile. The crude was quantified by Q-NMR to give a strength (w/w) of 16% and therefore the reaction gave a chemical yield of 32%. The crude was purified by chromatography column to give 44 mg of the pure, desired product 1-(4-nitrophenyl)cyclopropanecarbonitrile (the isolated yield is 30%)

Alternatively, the title product can be made from cyclisation of the isolated intermediate 2-[2-(4-nitrophenyl)sulfanylethyl]propanedinitrile (example 32) following this protocol

In a 3 necked round bottom flask equipped with magnetic bar, thermometer and nitrogen inlet was charged THF (5 mL). To this was added 2-[2-(4-nitrophenyl)sulfanylethyl]propanedinitrile (0.5 g, 2 mmol). The base Sodium tert-butanolate (1 equiv., 2 mmol) was added. The reaction was stirred one hour at this temperature, then 10 ml of ice cold water were added. The reaction was further acidified by 2 ml of 2N HCl—a solid precipitated out. The reaction was further diluted with 10 mL of water. The solid precipitate was filtered off on Buchner funnel under vacuum and the solid was washed with 15 mL of water before being collected. The white solid obtained was dried under reduced pressure at 50° C. 0.354 g of the pure, desired product 1-(4-nitrophenyl)cyclopropanecarbonitrile was obtained (Purity measured by Q-NMR being 94% (w/w) the isolated yield is 90%) m.p.=145.9-147.1° C. gradual decomposition up to 147° C.; ¹H NMR (CDCl₃, 400 MHz) δ (ppm)=8.22 (d, J=8.93 Hz, 2H), 7.43 (d, J=8.8 Hz, 2H), 1.94-1.86 (m, 2H), 1.57-1.49 (m, 2H): ¹³C NMR (CDCl₃, 101 MHz) δ (ppm)=147.2, 143.5, 126.0, 124.2, 121.1, 19.9, 14.1: IR (ATR, Diamond): v (cm⁻¹)=3108 (w), 3086 (w), 2237 (w), 1599 (m), 1515 (vs), 1346 (vs), 862 (m), 748 (m)

### Example 32: Preparation of 2-[2-(4-nitrophenyl)sulfanylethyl]propanedinitrile

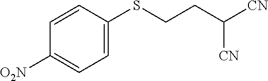 [SMILES: N#CC(C#N)CCSc1ccc([N+](=O)[O-])cc1]

In a 3 necked round bottom flask equipped with magnetic bar, thermometer and nitrogen inlet was charged DMF (10 mL) To this were added 5-amino-2,3-dihydrothiophene-4-carbonitrile (940 mg, 1.04 equiv., 7.4 mmol) and 1-fluoro-4-nitro-benzene (1 g, 1 equiv., 7.087 mmol). The mixture was cooled down at −40° C., Sodium tert-butanolate (2.03 equiv., 14.4 mmol) was added at this temperature. The reaction was stirred for 1 hour at this temperature, then 5 ml of ice cold water were added and the mixture was acidified with 10 mL of ice cold 1N HCl. The aqueous layer was extracted thrice with 20 mL of EtOAc, the combined organic layers were washed with 20 mL of brine and dried over solid Na₂SO₄ and filtered. The solvents were removed under reduced pressure to deliver 2.5 g of the crude desired product 2-[2-(4-nitrophenyl)sulfanylethyl]propanedinitrile as a yellow liquid which slowly solidified. Addition of 10 mL of water and stirring led to a suspension which was filtered on a Buchner funnel and the solid was collected after having being washed with 15 mL of water. The solid was then dried under vacuum at 50° C.; 1.25 g of a yellowish solid was then obtained and quantified by Q-NMR to give a strength (w/w) of 72% and therefore the reaction gave a chemical yield of 51%. ¹H NMR (D6-DMSO, 400 MHz) δ (ppm)=8.16 (2H, d, J=8.9 Hz, 2H), 7.57 (2H, d, J=8.8 Hz), 4.95 (1H, t, J=7 Hz), 3.29 (2H, t, J=6.5 Hz), 2.4 (2H, q, J=6.7 Hz)

### Example 33: Preparation of 1-(6-chloroquinoxalin-2-yl)cyclopropanecarbonitrile

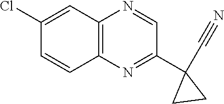 [SMILES: N#CC1(c2cnc3cc(Cl)ccc3n2)CC1]

In a 3 necked round bottom flask equipped with magnetic bar, thermometer and nitrogen inlet was charged DMF (10 mL). To this were added 5-amino-2,3-dihydrothiophene-4-carbonitrile (634 mg, 1 equiv., 5.02 mmol) and 2,6-dichloroquinoxaline (1 g, 1 equiv., 5.02 mmol). Sodium tert-butanolate (2.2 equiv., 11.1 mmol) was added at room temperature. The reaction was stirred for 3 hours then 10 ml of water were added. The reaction was further acidified by 5 ml of 2N HCl while a solid was precipitating out. The solid precipitate was filtered off on Buchner funnel under vacuum, the brownish solid obtained was dried under reduced pressure at 50° C. 1.71 g of the crude, desired product 1-(6-chloroquinoxalin-2-yl)cyclopropanecarbonitrile was obtained (Purity measured by Q-NMR being 47% (w/w) the chemical yield is 70%). The crude was purified by chromatography column to give 0.79 g of the pure, desired product 1-(6-chloroquinoxalin-2-yl)cyclopropanecarbonitrile as a white solid (Purity measured by Q-NMR being 98% (w/w) the isolated yield is 67%)

Alternatively, the title product can be made from cyclisation of the isolated intermediate 2-[2-(6-chloroquinoxalin-2-yl)sulfanylethyl]propanedinitrile (example 34) following this protocol.

In a 3 necked round bottom flask equipped with magnetic bar, thermometer and nitrogen inlet was charged THF (5 mL). To this was added 2-[2-(6-chloroquinoxalin-2-yl)sulfanylethyl]propanedinitrile (500 mg, 1 equiv., 1.73 mmol). Sodium tert-butanolate (1 equiv., 1.73 mmol) was added at room temperature The reaction was stirred for 1 hour then 10 ml of ice cold water were added. The reaction was further acidified by 2 ml of 2N HCl while a solid had precipitated out. The mixture was further diluted by 10 mL of water, the solid precipitate was filtered off on Buchner funnel under vacuum and rinsed with 15 mL of water; the white solid obtained was dried under reduced pressure at 50° C. 0.364 g of the pure, desired product 1-(6-chloroquinoxalin-2-yl)cyclopropanecarbonitrile was obtained (Purity measured by Q-NMR being 93% (w/w) the isolated yield is 85%) ¹H NMR (CDCl₃, 400 MHz) δ (ppm)=1.89-1.96 (m, 2H), 1.99-2.06 (m, 2H), 7.71 (dd, J=9.05, 2.32 Hz, 1H), 7.89 (d, J=9.05 Hz, 1H), 8.12 (d, J=2.32 Hz, 1H), 9.27 (s, 1H).

### Example 34: Preparation of 2-[2-(6-chloroquinoxalin-2-yl)sulfanylethyl]propanedinitrile

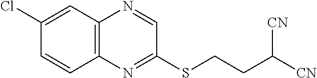 [SMILES: N#CC(C#N)CCSc1cnc2cc(Cl)ccc2n1]

In a 3 necked round bottom flask equipped with magnetic bar, thermometer and nitrogen inlet was charged DMF (10 mL). To this were added 5-amino-2,3-dihydrothiophene-4-carbonitrile (634 mg, 1 equiv., 5.02 mmol) and 2,6-dichloroquinoxaline (1 g, 1 equiv., 5.02 mmol). The reaction mixture was cooled down to −40° C. and Sodium tert-butanolate (2.2 equiv., 11.1 mmol) was added at this temperature The reaction was stirred one hour at this temperature. 5 mL of ice-cold water were added, followed by 10 mL of ice cold 2N HCl; the aqueous layer was extracted thrice with 20 mL of EtOAc, the combined organic layers were washed 20 mL of brine, before being dried over solid Na₂SO₄ and filtered. The solvents were removed under reduced pressure to deliver 1.35 g of the crude desired product 2-[2-(6-chloroquinoxalin-2-yl)sulfanylethyl]propanedinitrile as an orange oil (Purity measured by Q-NMR being 85% (w/w) the chemical yield is 79%). The crude was purified by chromatography column to give 0.99 g of the pure, desired product 2-[2-(6-chloroquinoxalin-2-yl)sulfanylethyl]propanedinitrile as a white solid (Purity measured by Q-NMR being 97% (w/w) the isolated yield is 66%). ¹H NMR (CDCl₃, 400 MHz) δ (ppm)=2.59 (q, J=6.92 Hz, 2H), 3.60 (t, J=6.88 Hz, 2H), 4.03 (t, J=7.00 Hz, 1H), 7.69 (d, J=8.51 Hz, 1H), 7.97 (d, J=8.88 Hz. 1H), 8.04 (d, J=2.38 Hz, 1H), 8.63 (s. 1H).

### Example 35: Preparation of 1-(3-chloropyrazin-2-yl)cyclopropanecarbonitrile

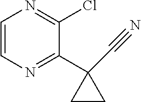 [SMILES: N#CC1(c2nccnc2Cl)CC1]

In a 3 necked round bottom flask equipped with magnetic bar, thermometer and nitrogen inlet was charged DMF (10 mL). To this were added 5-amino-2,3-dihydrothiophene-4-carbonitrile (847 mg, 1 equiv., 6.71 mmol) and 2,3-dichloropyrazine (1 g, 1 equiv., 6.71 mmol). Sodium tert-butanolate (2.2 equiv., 14.8 mmol) was added at room temperature, then the reaction was heated up to 65° C., stirred for nine hours at this temperature, and overnight at room temperature. 10 mL of water were added, followed by 10 mL of 1N HCl; the aqueous layer was extracted thrice with 50 mL of EtOAc, the combined organic layers were washed 20 mL of brine, before being dried over solid Na₂SO₄ and filtered. The solvents were removed under reduced pressure to deliver 2.77 g of the crude desired product 1-(3-chloropyrazin-2-yl)cyclopropanecarbonitrile as an orange liquid. (Purity measured by Q-NMR being 33% (w/w) the chemical yield is 77%). The crude was purified by chromatography column to give 0.75 g of relatively pure, desired product 1-(3-chloropyrazin-2-yl)cyclopropanecarbonitrile as a yellow liquid (Purity measured by Q-NMR being 84% (w/w) the isolated yield is 52%). Higher purity can be achieved by proceeding to an extra column chromatography.

Alternatively, the title product can be made from cyclisation of the isolated intermediate 2-[2-(3-chloropyrazin-2-yl)sulfanylethyl]propanedinitrile (example 36) following this protocol

In a 3 necked round bottom flask equipped with magnetic bar, thermometer and nitrogen inlet was charged THF (5 mL). To this was added 2-[2-(3-chloropyrazin-2-yl)sulfanylethyl]propanedinitrile (500 mg, 1 equiv., 2.09 mmol). Sodium tert-butanolate (1 equiv., 2.09 mmol) was added at room temperature, then the reaction was heated up to 65° C. and stirred for four hours at this temperature, before being allowed to cool down to room temperature. 5 mL of water were added, followed by 10 mL of 1N HCl; the aqueous layer was extracted thrice with 20 mL of EtOAc, the combined organic layers were washed 20 mL of brine, before being dried over solid Na₂SO₄ and filtered. The solvents were removed under reduced pressure to deliver 0.52 g of the crude desired product 1-(3-chloropyrazin-2-yl)cyclopropanecarbonitrile as an orange liquid. (Purity measured by Q-NMR being 45% (w/w) the chemical yield is 62%) The crude was purified by chromatography column to give 0.22 g of pure, desired product 1-(3-chloropyrazin-2-yl)cyclopropanecarbonitrile as a colorless liquid (Purity measured by Q-NMR being 94% (w/w) the isolated yield is 55%). ¹H NMR (CDCl₃, 400 MHz) δ (ppm)=1.68-1.73 (m, 2H), 1.80-1.85 (m, 2H), 8.38 (d, J=2.38 Hz, 1H), 8.44 (d, J=2.38 Hz, 1H).

### Example 36: Preparation of 2-[2-(3-chloropyrazin-2-yl)sulfanylethyl]propanedinitrile

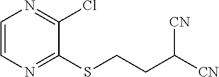 [SMILES: N#CC(C#N)CCSc1nccnc1Cl]

In a 3 necked round bottom flask equipped with magnetic bar, thermometer and nitrogen inlet was charged DMF (10 mL). To this were added 5-amino-2,3-dihydrothiophene-4-carbonitrile (860 mg, 1 equiv., 6.7 mmol) and 2,3-dichloropyrazine (1 g, 1 equiv., 6.7 mmol). The reaction mixture was cooled down to −40° C. and Sodium tert-butanolate (2.2 equiv., 15 mmol) was added at this temperature. The reaction was stirred two hours at −40° C., then 5 mL of ice-cold water were added, followed by 10 mL of ice-cold 1N HCl; the solid precipitate was then filtered off on Buchner funnel under vacuum and rinsed with 15 mL of water; the yellow solid obtained was dried under reduced pressure at 50° C. 1.4 g of the pure, desired product 2-[2-(3-chloropyrazin-2-yl)sulfanylethyl]propanedinitrile. Purity measured by Q-NMR being 95% (w/w) the isolated yield is 83% ¹H NMR (CDCl₃, 400 MHz) δ (ppm)=2.50 (q, J=7.13 Hz, 2H), 3.44 (t, J=6.88 Hz, 2H), 4.00 (t, J=7.25 Hz, 1H), 8.13 (d, J=2.63 Hz, 1H), 8.36 (d, J=2.63 Hz, 1H).

### Example 37: Preparation of Diversely Substituted 5-amino-2,3-dihydrothiophene-4-carbonitriles

The following nine diversely substituted 5-amino-2,3-dihydrothiophene-4-carbonitriles: 5-amino-2-[(4-chlorophenoxy)methyl]-2,3-dihydrothiophene-4-carbonitrile; 5-amino-2-(phenoxymethyl)-2,3-dihydrothiophene-4-carbonitrile; 5-amino-2-hexyl-2,3-dihydrothiophene-4-carbonitrile; 5-amino-2-phenyl-2,3-dihydrothiophene-4-carbonitrile as a mixture with 5-amino-3-phenyl-2,3-dihydrothiophene-4-carbonitrile; 5-amino-2,2-diethyl-3H-thiophene-4-carbonitrile; 5-amino-3-ethyl-2-heptyl-2,3-dihydrothiophene-4-carbonitrile; and 5-amino-3-ethyl-2-phenyl-2,3-dihydrothiophene-4-carbonitrile in a mixture with 5-amino-2-ethyl-3-phenyl-2,3-dihydrothiophene-4-carbonitrile: were made simply from corresponding epoxides and potassium thiocyanate (KSCN) (Scheme 4),

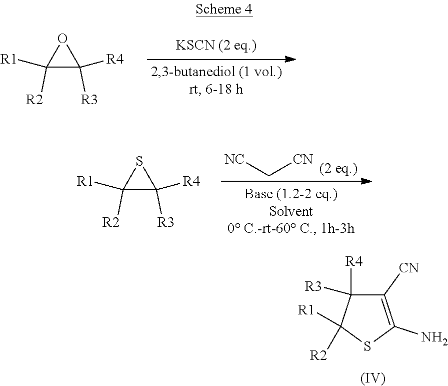

as described by Naoto Aoyagi et al in ChemistrySelect, 2, 4466-4468, 2017, followed by treatment with malonitrile and a base as NaH as described in Yamagata, Kenji et al in Chemical & Pharmaceutical Bulletin, 30(12), 4396-401; 1982: (NMP as solvent instead of DMSO for safety reasons) or by LiOH·H₂O (in THF or MeTHF as solvent) or alternatively, by NaH in DME as solvent as described in Wamhoff, Heinrich and Thiemig, Heinz Albrecht in Chemische Berichte, 118(11), 4473-85; 1985 In scheme 4, R₁-R₄ are as defined above. They were used as starting materials in the preparation of examples 38 to 51.

### Example 38: Preparation of 2-[2-[(4-chlorophenoxy)methyl]-1-cyano-cyclopropyl]pyridine-3-carbonitrile

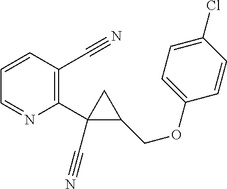 [SMILES: N#Cc1cccnc1C1(C#N)CC1COc1ccc(Cl)cc1]

In a 3 necked round bottom flask equipped with magnetic bar, thermometer and nitrogen inlet was charged DMF (5 mL). To this were added 5-amino-2-[(4-chlorophenoxy)methyl]-2,3-dihydrothiophene-4-carbonitrile (481 mg, 1 equiv., 1.80 mmol) and 2-chloropyridine-3-carbonitrile (0.25 g, 1 equiv., 1.80 mmol). Sodium tert-butanolate (2.2 equiv., 3.97 mmol) was added at room temperature, then the reaction was stirred for four hours at this temperature. 5 mL of ice cold water were added, followed by 10 mL of 1N HCl; the aqueous layer was extracted thrice with 30 mL of EtOAc, the combined organic layers were washed twice with 20 mL of brine, before being dried over solid Na₂SO₄ and filtered The solvents were removed under reduced pressure to deliver 4.4 g of the crude desired product 2-[2-[(4-chlorophenoxy)methyl]-1-cyano-cyclopropyl]pyridine-3-carbonitrile as an orange liquid. (Purity measured by Q-NMR being 6% (w/w) for the major diastereoisomer and 2% for the minor diastereoisomer, the total chemical yield is 58% (45% for the major and 13% for the minor diastereoisomer). The crude was purified by chromatography column to give 0.19 g of pure, desired product 2-[2-[(4-chlorophenoxy)methyl]-1-cyano-cyclopropyl]pyridine-3-carbonitrile (Major Isomer) and 54 mg of the minor diastereoisomer of the title compound as white solids (Purity measured by Q-NMR being 97% (w/w) for both fractions, the total isolated yield is 43% (33% Major and 10% minor isomer). Alternatively, the title product can be made from cyclisation of an isolated intermediate 2-[3-(4-chlorophenoxy)-2-[(3-cyano-2-pyridyl)sulfanyl]propyl]propanedinitrile (example 39) following this protocol. In a 3 necked round bottom flask equipped with magnetic bar, thermometer and nitrogen inlet was charged THF (5 mL). To this was added 2-[3-(4-chlorophenoxy)-2-[(3-cyano-2-pyridyl)sulfanyl]propyl]propanedinitrile (250 mg, 1 equiv., 0.68 mmol). Sodium tert-butanolate (1 equiv., 0.68 mmol) was added at room temperature, then the reaction was stirred for three hours at this temperature. The reaction mixture was poured onto 5 mL of aqueous saturated NH₄Cl, stirred for 10 minutes and the aqueous layer was extracted thrice with 20 mL of EtOAc. The combined organic layers were dried over solid Na₂SO₄ and filtered. The solvents were removed under reduced pressure to deliver 0.2 g of the crude desired product 2-[2-[(4-chlorophenoxy)methyl]-1-cyano-cyclopropyl]pyridine-3-carbonitrile as an orange liquid. (Purity measured by Q-NMR being 45% (w/w) for the major diastereoisomer and 18% for the minor diastereoisomer, the total chemical yield is 60%. The crude was purified by chromatography column to give 72 mg of pure, desired product 2-[2-[(4-chlorophenoxy)methyl]-1-cyano-cyclopropyl]pyridine-3-carbonite (Major Isomer) and in another fraction 32 mg of the minor diastereoisomer of the title compound as yellow solids (the total isolated yield is 47% (34% Major and 13% minor isomer). ¹H NMR (CDCl₃, 400 MHz) δ (ppm)=for the Major Fraction 1.87 (dd, J=7.44, 5.69 Hz, 1H), 2.21 (dd, J=9.01, 5.63 Hz, 1H), 2.39-2.47 (m, 1H), 4.29-4.4 (m, 2H), 6.93 (d, J=8 Hz, 2H), 7.26 (d, J=8 Hz, 2H), 7.43 (dd, J=7.75, 4.88 Hz, 1H), 8.06 (dd, J=7.82, 1.81 Hz, 1H), 8.73 (dd, J=4.88, 1.75 Hz, 1H). ¹H NMR (CDCl₃, 400 MHz) δ (ppm)=for the minor Fraction 1.99 (dd, J=9.35, 5.94 Hz, 1H), 2.46 (dd, J=7.60, 5.97 Hz, 1H), 2.57-2.73 (m, 1H), 3.29 (dd, J=10.68, 8.33 Hz, 1H), 4.11 (dd, J=10.69, 4.41 Hz, 1H), 6.53-6.59 (m, 2H), 7.09-7.13 (m, 2H), 7.34 (dd, J=7.85, 4.91 Hz, 1H), 7.95 (dd, J=7.85, 1.75 Hz, 1H), 8.67 (dd, J=4.91, 1.75 Hz, 1H). 2D-NMR's (including NOESY) did show that the major isomer is the racemic mixture of 2-[(1S,2R)-2-[(4-chlorophenoxy)methyl]-1-cyano-cyclopropyl]pyridine-3-carbonitrile and its enantiomer; whereas the minor isomer is the racemic mixture of 2-[(1R,2R)-2-[(4-chlorophenoxy)methyl]-1-cyano-cyclopropyl]pyridine-3-carbonitrile and its enantiomer.

### Example 39: Preparation of 2-[3-(4-chlorophenoxy)-2-[(3-cyan-2-pyridyl)sulfanyl]propyl]propanedinitrile

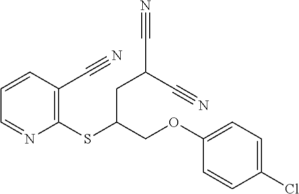 [SMILES: N#Cc1cccnc1SC(COc1ccc(Cl)cc1)CC(C#N)C#N]

In a 3 necked round bottom flask equipped with magnetic bar, thermometer and nitrogen inlet was charged DMF (5 mL). To this were added 5-amino-2-[(4-chlorophenoxy)methyl]-2,3-dihydrothiophene-4-carbonite (481 mg, 1 equiv., 1.80 mmol) and 2-chloropyridine-3-carbonitrile (0.25 g, 1 equiv., 1.80 mmol). The reaction mixture was cooled down to −40° C. and Sodium tert-butanolate (2.2 equiv., 3.97 mmol) was added at this temperature. The reaction was stirred one hour at this temperature. 5 mL of ice-cold water were added, followed by 10 mL of ice cold 1N HCl; the aqueous layer was extracted thrice with 20 mL of EtOAc, the combined organic layers were washed 20 mL of brine, before being dried over solid Na₂SO₄ and filtered The solvents were removed under reduced pressure to deliver 1.59 g of the crude desired product 2-[3-(4-chlorophenoxy)-2-[(3-cyano-2-pyridyl)sulfanyl]propyl]propanedinitrile as an orange liquid. (Purity measured by Q-NMR being 47% (w/w) the chemical yield is 75%). The crude was purified by chromatography column to give 0.38 g of pure, desired product 2-[3-(4-chlorophenoxy)-2-[(3-cyano-2-pyridyl)sulfanyl]propyl]propanedinitrle as a white solid (Purity measured by Q-NMR being 94% (w/w) the isolated yield is 53%). ¹H NMR (CDCl₃, 400 MHz) δ (ppm)=2.52 (ddd, J=14.49, 9.96, 5.62 Hz, 1H), 2.79 (ddd, J=14.49, 9.84, 4.77 Hz, 1H), 4.11-4.18 (m, 2H), 4.29 (dd, J=9.72, 3.73 Hz, 1H), 4.59-4.67 (m, 1H), 6.78-6.83 (m, 2H), 7.14-7.22 (m, 3H), 7.83 (dd, J=7.76, 1.77 Hz, 1H), 8.58 (dd, J=4.89, 1.71 Hz, 1H)

### Example 40: Preparation of 2-[1-cyano-2-(phenoxymethyl)cyclopropyl]pyridine-3-carbonitrile

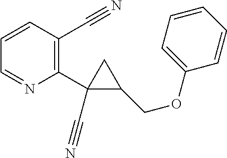 [SMILES: N#Cc1cccnc1C1(C#N)CC1COc1ccccc1]

In a 3 necked round bottom flask equipped with magnetic bar, thermometer and nitrogen inlet was charged DMF (5 mL). To this were added 5-amino-2-(phenoxymethyl)-2,3-dihydrothiophene-4-carbonitrile (420 mg, 1 equiv., 1.80 mmol) and 2-chloropyridine-3-carbonitrile (0.25 g, 1 equiv., 1.80 mmol). Sodium tert-butanolate (2.2 equiv., 4 mmol) was added at room temperature, then the reaction was stirred for four hours at this temperature. 5 mL of ice cold water were added, followed by 10 mL of 1N HCl; the aqueous layer was extracted thrice with 30 mL of EtOAc, the combined organic layers were washed with 20 mL of brine, before being dried over solid Na₂SO₄ and filtered. The solvents were removed under reduced pressure to deliver 0.64 g of the crude desired product 2-[1-cyano-2-(phenoxymethyl)cyclopropyl]pyridine-3-carbonitrile as an orange liquid. (Purity measured by Q-NMR being 36% (w/w) for the major diastereoisomer and 11% for the minor diastereoisomer, the total chemical yield is 60% (46% for the major and 14% for the minor diastereoisomer) The crude was purified by chromatography column to give 0.23 g of pure, desired product 2-[1-cyano-2-(phenoxymethyl)cyclopropyl]pyridine-3-carbonitrile (Major Isomer) and in another fraction 70 mg of the minor diastereoisomer of the title compound as white solids (Purity measured by Q-NMR being >95% (w/w) for both fractions, the total isolated yield is 54% (44% Major and 10% minor isomer).

Alternatively, the title product can be made from cyclisation of an isolated intermediate 2-[2-[(3-cyano-2-pyridyl)sulfanyl]-3-phenoxy-propyl]propanedinitrile (example 41) following this protocol.

In a 3 necked round bottom flask equipped with magnetic bar, thermometer and nitrogen inlet was charged THF (4 mL) To this was added 2-[2-[(3-cyano-2-pyridyl)sulfanyl]-3-phenoxy-propyl]propanedinitrile (250 mg, 1 equiv., 0.75 mmol). Sodium tert-butanolate (1 equiv., 0.75 mmol) was added at room temperature, then the reaction was heated up to 65° C. and stirred at this temperature for three hours. The reaction mixture was allowed to cool down to room temperature and poured onto 5 mL of aqueous saturated NH₄Cl, stirred for 10 minutes and the aqueous layer was extracted thrice with 20 mL of EtOAc. The combined organic layers were dried over solid Na₂SO₄ and filtered. The solvents were removed under reduced pressure to deliver 0.21 g of the crude desired product 2-[1-cyano-2-(phenoxymethyl)cyclopropyl]pyridine-3-carbonite as an orange liquid. (Purity measured by Q-NMR being 29% (w/w) for the major diastereoisomer and 10% for the minor diastereoisomer, the total chemical yield is 54% (39% for the major and 15% for the minor diastereoisomer). The crude was purified by chromatography column to give 60 mg of pure, desired product 2-[1-cyano-2-(phenoxymethyl)cyclopropyl]pyridine-3-carbonitrile (Major Isomer) and in another fraction 20 mg of the minor diastereoisomer of the title compound as pale yellow solids (the total isolated yield is 42% (30% in the Major isomer and 12% for the minor isomer). ¹H NMR (CDCl₃, 400 MHz) δ (ppm)=for the Major Fraction 1.89 (dd, J=7.32, 5.82 Hz, 1H), 2.21 (dd, J=9.01, 5.63 Hz, 1H), 2.39-2.53 (m, 1H), 4.25-4.34 (m, 1H), 4.45 (dd, J=10.44, 6.82 Hz, 1H), 6.97-7.06 (m, 3H), 7.21-7.39 (m, 2H), 7.42 (dd, J=7.85, 4.88 Hz, 1H) 8.04 (dd, J=7.85, 1.75 Hz, 1H) 8.73 (dd, J=4.88, 1.75 Hz, 1H). ¹H NMR (CDCl₃, 400 MHz) δ (ppm)=for the minor Fraction 2.10 (dd, J=9.36, 5.89 Hz, 1H), 2.55-2.60 (m, 1H), 2.68-2.86 (m, 1H), 3.49 (dd, J=10.71, 8.05 Hz, 1H), 4.23 (dd, J=10.74, 4.42 Hz, 1H), 6.71-6.76 (m, 2H), 6.89-7.09 (m, 1H), 7.24-7.29 (m, 2H), 7.43 (ddd, J=7.84, 4.90, 0.89 Hz, 1H), 8.05 (d, J=7.80 Hz, 1H). 8.78 (d, J=4.96 Hz. 1H)

### Example 41: Preparation of 2-[2-[(3-cyano-2-pyridyl)sulfanyl]-3-phenoxy-propyl]propanedinitrile

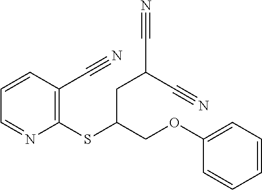 [SMILES: N#Cc1cccnc1SC(COc1ccccc1)CC(C#N)C#N]

In a 3 necked round bottom flask equipped with magnetic bar, thermometer and nitrogen inlet was charged DMF (5 mL). To this were added 5-amino-2-(phenoxymethyl)-2,3-dihydrothiophene-4-carbonitrile (419 mg, 1 equiv., 1.80 mmol) and 2-chloropyridine-3-carbonitrile (0.25 g, 1 equiv., 1.80 mmol). The reaction mixture was cooled down to −40° C. and Sodium tert-butanolate (2.2 equiv., 3.97 mmol) was added at this temperature. The reaction was stirred 1.5 hour at this temperature. 5 mL of ice-cold water were added, followed by 10 mL of ice cold 1N HCl; the aqueous layer was extracted thrice with 20 mL of EtOAc, the combined organic layers were washed 20 mL of brine, before being dried over solid Na₂SO₄ and filtered. The solvents were removed under reduced pressure to deliver 1.9 g of the crude desired product 2-[2-[(3-cyano-2-pyridyl)sulfanyl]-3-phenoxy-propyl]propanedinitrile as an orange liquid. (Purity measured by Q-NMR being 23% (w/w) the chemical yield is 72%). The crude was purified by chromatography column to give 0.47 g of pure, desired product 2-[2-[(3-cyano-2-pyridyl)sulfanyl]-3-phenoxy-propyl]propanedinitrile as a pale yellow solid (Purity measured by Q-NMR being 72% (w/w) the isolated yield is 56%). ¹H NMR (CDCl₃, 400 MHz) δ (ppm)=2.61 (ddd, J=14.70, 9.44, 5.75 Hz, 1H), 2.85-2.93 (m, 1H), 4.19-4.28 (m, 2H), 4.33-4.48 (m, 1H), 4.63-4.80 (m, 1H), 6.95 (d, J=8.38 Hz, 2H), 7.02 (t, J=7.19 Hz, 1H), 7.22 (dd, J=7.69, 4.94 Hz, 1H), 7.30-7.37 (m, 2H), 7.90 (dd, J=7.75, 1.38 Hz, 1H), 8.63-8.68 (m, 1H).

### Example 42: Preparation of 2-(1-cyano-2-hexyl-cyclopropyl)pyridine-3-carbonitrile

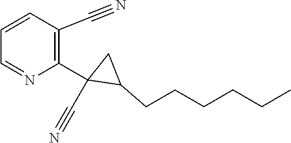 [SMILES: CCCCCCC1CC1(C#N)c1ncccc1C#N]

In a 3 necked round bottom flask equipped with magnetic bar, thermometer and nitrogen inlet was charged DMF (5 mL). To this were added 5-amino-2-hexyl-2,3-dihydrothiophene-4-carbonitrile (759 mg, 1 equiv., 3.61 mmol) and 2-chloropyridine-3-carbonitrile (0.5 g, 1 equiv, 3.61 mmol). Sodium tert-butanolate (2.2 equiv., 7.94 mmol) was added at room temperature, then the reaction was stirred for two hours at this temperature. 10 mL of ice cold water were added, followed by 5 mL of 2N HCl; the aqueous layer was extracted thrice with 30 mL of EtOAc, the combined organic layers were washed with 20 mL of brine, before being dried over solid Na₂SO₄ and filtered. The solvents were removed under reduced pressure to deliver 1.68 g of the crude desired product 2-(1-cyano-2-hexyl-cyclopropyl)pyridine-3-carbonitrile as an orange liquid. (Purity measured by Q-NMR being 30% (w/w) for the major diastereoisomer and 10% for the minor diastereoisomer, the total chemical yield is 74% (55.5% for the major and 18.5% for the minor diastereoisomer). The crude was purified by chromatography column to give 0.265 g of pure (91% w/w as measured by QNMR), desired product 2-(1-cyano-2-hexyl-cyclopropyl)pyridine-3-carbonitrile (Major Isomer) and in another fraction 52 mg of a mixture of major and minor diastereoisomers of the title compound in a 2:1 ratio. The purity measured for the minor fraction is, by Q-NMR, around 60% (w/w) for the total of both isomers, the overall isolated yield from both fractions combined is 66% (54% Major and 12% minor isomer).

Alternatively, the title product can be made from cyclisation of an isolated intermediate 2-[2-[(3-cyano-2-pyridyl)sulfanyl]octyl]propanedinitrile (example 43) following this protocol. In a 3 necked 25 mL Round bottom flask equipped with magnetic bar, thermometer and nitrogen inlet was charged THF (5 mL). To this was added 2-[2-[(3-cyano-2-pyridyl)sulfanyl]octyl]propanedinitrile (500 mg, 1 equiv., 1.60 mmol). Sodium tert-butanolate (1 equiv., 1.60 mmol) was added at room temperature, then the reaction was stirred at this temperature for two hours. The reaction mixture was poured onto 2 mL of 2N HCl, stirred for 10 minutes and the aqueous layer was extracted thrice with 20 mL of EtOAc. The combined organic layers were dried over solid Na₂SO₄ and filtered. The solvents were removed under reduced pressure to deliver 0.37 g of the crude desired product 2-(1-cyano-2-hexyl-cyclopropyl)pyridine-3-carbonitrile as an orange liquid. (Purity measured by Q-NMR being 55% (w/w) for the major diastereoisomer and 28% for the minor diastereoisomer, the total chemical yield is 76% (50% for the major and 26% for the minor diastereoisomer). The crude was purified by chromatography column to give three fractions. First fraction (20 mg) is the minor isomer only (5% Chemical yield for this fraction), the second fraction (155 mg) is a mixture of the two isomers (Purity measured by Q-NMR is 59% (w/w) for the major diastereoisomer and 33% for the minor diastereoisomer, the chemical yield in this fraction is 34% (22% for the major and 12% for the minor diastereoisomer), and the third fraction (69 mg) is the major isomer only (17% Chemical yield for this fraction) The total, combined fractions, isolated yield is thus 56% overall. ¹H NMR (CDCl₃, 400 MHz) δ (ppm)=for the major isomer 0.88-0.91 (m, 3H), 1.24-1.44 (m, 6H), 1.53-1.72 (m, 5H), 1.84-2.00 (m, 2H), 2.11 (dd, J=8.66, 5.14 Hz, 1H), 7.38 (dd, J=7.88, 4.88 Hz, 1H), 8.02 (dd, J=7.78, 1.76 Hz, 1H), 8.70 (dd, J=4.77, 1.76 Hz, 1H). ¹H NMR (CDCl₃, 400 MHz) δ (ppm)=for the minor isomer 0.48-0.54 (m, 1H), 0.83 (t, J=7.03 Hz, 3H), 1.10-1.28 (m, 6H), 1.31-1.46 (m, 3H), 1.86 (dd, J=9.03, 5.52 Hz, 1H), 2.05 (dd, J=7.78, 5.52 Hz, 1H), 2.12-2.16 (m, 1H), 7.43 (dd, J=7.91, 4.89 Hz, 1H), 8.04 (dd, J=7.78, 1.76 Hz, 1H), 8.75 (dd, J=4.77, 1.76 Hz, 1H). 2D-NMR's (including NOESY) did show that the major isomer is the racemic mixture of 2-[(1R,2S)-1-cyano-2-hexyl-cyclopropyl]pyridine-3-carbonitrile and its enantiomer; whereas the minor isomer is the racemic mixture of 2-[(1R,2R)-1-cyano-2-hexyl-cyclopropyl]pyridine-3-carbonitrile and its enantiomer.

### Example 43: Preparation of 2-[2-[(3-cyano-2-pyridyl)sulfanyl]octyl]propanedinitrile

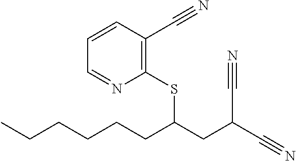 [SMILES: CCCCCCC(CC(C#N)C#N)Sc1ncccc1C#N]

In a 3 necked round bottom flask equipped with magnetic bar, thermometer and nitrogen inlet was charged DMF (5 mL) To this were added 5-amino-2-hexyl-2,3-dihydrothiophene-4-carbonitrile (759 mg, 1 equiv., 3.61 mmol) and 2-chloropyridine-3-carbonitrile (0.5 g, 1 equiv., 3.61 mmol). The reaction mixture was cooled down to −40° C. and Sodium tert-butanolate (2.2 equiv., 3.97 mmol) was added at this temperature. The reaction was stirred 4.5 hours at this temperature. 5 mL of ice-cold water were added, followed by 10 mL of ice cold 2N HCl; the aqueous layer was extracted thrice with 50 mL of EtOAc, the combined organic layers were washed 20 mL of brine, before being dried over solid Na₂SO₄ and filtered. The solvents were removed under reduced pressure to deliver 1.6 g of the crude desired product 2-[2-[(3-cyano-2-pyridyl)sulfanyl]octyl]propanedinitrle as an orange liquid. (Purity measured by Q-NMR being 50% (w/w) the chemical yield is 70%). The crude was purified by chromatography column to give 0.75 g of pure, desired product 2-[2-[(3-cyano-2-pyridyl)sulfanyl]octyl]propanedinitrile as a pale yellow liquid (Purity measured by Q-NMR being 97% (w/w) the isolated yield is 65%). ¹H NMR (CDCl₃, 400 MHz) δ (ppm)=0.82-0.94 (m, 3H), 1.24-1.37 (m, 6H), 1.43-1.60 (m, 2H), 1.83 (q, J=7.30 Hz, 2H), 2.42 (ddd, J=14.51, 9.69, 5.19 Hz, 1H), 2.57 (ddd, J=14.26, 10.01, 4.13 Hz, 1H), 4.10 (dd, J=10.01, 5.13 Hz, 1H), 4.21-4.29 (m, 1H), 7.18 (dd, J=7.75, 4.88 Hz, 1H), 7.87 (dd, J=7.75, 1.75 Hz, 1H), 8.62 (dd, J=4.88, 1.75 Hz, 1H)

### Example 44: Preparation of 2-(1-cyano-2-phenyl-cyclopropyl)pyridine-3-carbonitrile

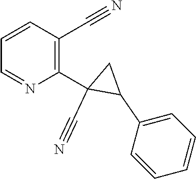 [SMILES: N#Cc1cccnc1C1(C#N)CC1c1ccccc1]

In a 3 necked round bottom flask equipped with magnetic bar, thermometer and nitrogen inlet was charged DMF (5 mL). To this were added a mixture of 5-amino-2-phenyl-2,3-dihydrothiophene-4-carbonitrile and 5-amino-3-phenyl-2,3-dihydrothiophene-4-carbonitrile in almost a 1:1 ratio (730 mg, 1 equiv., 3.61 mmol) and 2-chloropyridine-3-carbonite (0.5 g, 1 equiv., 3.61 mmol). Sodium tert-butanolate (2.2 equiv., 7.94 mmol) was added at room temperature, then the reaction was stirred for two hours at this temperature. 10 mL of ice cold water were added, followed by 10 mL of 2N HCl: the aqueous layer was extracted thrice with 30 mL of EtOAc, the combined organic layers were washed with 20 mL of brine, before being dried over solid Na₂SO₄ and filtered. The solvents were removed under reduced pressure to deliver 2.3 g of the crude desired product 2-(1-cyano-2-phenyl-cyclopropyl)pyridine-3-carbonitrile as an orange liquid. (Purity measured by Q-NMR being 16% (w/w) for the major diastereoisomer and 2.1% for the minor diastereoisomer, the total chemical yield is 46% (40% for the major and 6% for the minor diastereoisomer). The crude was purified by chromatography column to give 0.3 g of pure (94% w/w as measured by QNMR), desired product 2-(1-cyano-2-phenyl-cyclopropyl)pyridine-3-carbonitrile (Major Isomer; isolated yield 30%) and in another fraction a mixture of major and minor diastereoisomers of the title compound. Alternatively, the title product can be made from cyclisation of isolated intermediates mixture consisting of 2-[2-[(3-cyano-2-pyridyl)sulfanyl]-2-phenyl-ethyl]propanedinitrile and 2-[2-[(3-cyano-2-pyridyl)sulfanyl]-1-phenyl-ethyl]propanedinitrile (example 45) following this protocol. In a 3 necked 25 mL Round bottom flask equipped with magnetic bar, thermometer and nitrogen inlet was charged THF (5 mL). To this were added a mixture consisting of 2-[2-[(3-cyano-2-pyridyl)sulfanyl]-2-phenyl-ethyl]propanedinitrile and 2-[2-[(3-cyano-2-pyridyl)sulfanyl]-1-phenyl-ethyl]propanedinitrile (500 mg, 1 equiv., 1.64 mmol). Sodium tert-butanolate (1 equiv., 1.64 mmol) was added at room temperature, then the reaction was stirred at this temperature overnight. The reaction mixture was poured onto 2 mL of 2N HCl, stirred for 10 minutes and the aqueous layer was extracted thrice with 20 mL of EtOAc. The combined organic layers were dried over solid Na₂SO₄ and filtered. The solvents were removed under reduced pressure to deliver 0.35 g of the crude desired product 2-(1-cyano-2-phenyl-cyclopropyl)pyridine-3-carbonitrile as an orange liquid. (Purity measured by Q-NMR being 56% (w/w) for the major diastereoisomer and 21% for the minor diastereoisomer, the total chemical yield is 67% (49% for the major and 18% for the minor diastereoisomer). The crude was purified by chromatography column to separate the two isomers (43% Isolated yield for the major isomer and 8% isolated yield for the minor isomer). The total, combined fractions, isolated yield is thus 51% overall. ¹H NMR (CDCl₃, 400 MHz) δ (ppm)=for the major isomer 2.31 (dd, J=8.00, 5.88 Hz, 1H), 2.48 (dd, J=9.11, 5.81 Hz, 1H), 3.19 (t, J=8.63 Hz, 1H), 7.33-7.54 (m, 6H), 8.07 (dd, J=7.82, 1.69 Hz, 1H), 8.78 (dd, J=4.89, 1.83 Hz, 1H) ¹H NMR (CDCl₃, 400 MHz) δ (ppm)=for the minor isomer 2.20 (dd, J=9.4, 6.4 Hz, 1H), 2.94 (dd, J=8.3, 6.5 Hz, 1H), 3.47 (t, J=8.9 Hz, 1H), 6.95-7.03 (m, 2H), 7.05-7.11 (m, 3H), 7.21 (dd, J=7.9, 4.9 Hz, 1H), 7.74 (dd, J=7.9, 1.8 Hz, 1H), 8.63 (dd, J=5.0, 1.8 Hz, 1H). 2D-NMR's (including NOESY) did show that the major isomer is the racemic mixture of 2-[(1S,2S)-1-cyano-2-phenyl-cyclopropyl]pyridine-3-carbonitrile and its enantiomer, whereas the minor isomer is the racemic mixture of 2-[(1R,2S)-1-cyano-2-phenyl-cyclopropyl]pyridine-3-carbonitrile and its enantiomer.

### Example 45: Preparation of 2-2-[(3-cyano-2-pyridyl)sulfanyl]-2-phenyl-ethyl]propanedinitrile and 2-[2-[(3-cyano-2-pyridyl)sulfanyl]-1-phenyl-ethyl]propanedinitrile

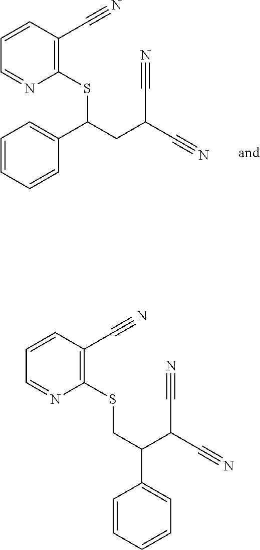 [SMILES: N#Cc1cccnc1SC(CC(C#N)C#N)c1ccccc1 ; N#Cc1cccnc1SCC(c1ccccc1)C(C#N)C#N]

In a 3 necked round bottom flask equipped with magnetic bar, thermometer and nitrogen inlet was charged DMF (5 mL). To this were added a mixture of 5-amino-2-phenyl-2,3-dihydrothiophene-4-carbonitrile and 5-amino-3-phenyl-2,3-dihydrothiophene-4-carbonitrile in almost a 1.1 ratio (730 mg, 1 equiv., 3.61 mmol) and 2-chloropyridine-3-carbonitrile (0.5 g, 1 equiv., 3.61 mmol). The reaction mixture was cooled down to −40° C. and Sodium tert-butanolate (2.2 equiv., 3.97 mmol) was added at this temperature. The reaction was stirred 2 hours at this temperature. 5 mL of ice-cold water were added, followed by 10 mL of ice cold 1N HCl; the aqueous layer was extracted thrice with 30 mL of EtOAc, the combined organic layers were washed 20 mL of brine, before being dried over solid Na₂SO₄ and filtered. The solvents were removed under reduced pressure to deliver 2.3 g of the crude desired products 2-[2-[(3-cyano-2-pyridyl)sulfanyl]-2-phenyl-ethyl]propanedinitrile and 2-[2-[(3-cyano-2-pyridyl)sulfanyl]-1-phenyl-ethyl]propanedinitrile as an orange liquid (Purity measured by Q-NMR being 31% (w/w) the chemical yield is 74%). The crude was purified by chromatography column to give a mixture (˜1-1) of 2-[2-[(3-cyano-2-pyridyl)sulfanyl]-2-phenyl-ethyl]propanedinitrile and 2-[2-[(3-cyano-2-pyridyl)sulfanyl]-1-phenyl-ethyl]propanedinitrile with a total isolated yield of 68% and as a pale yellow liquid. ¹H NMR (CDCl₃, 400 MHz) δ (ppm) of the mixture=2.67-2.76 (m, 1H), 2.94-3.01 (m, 1H), 3.60-3.72 (m. 2H), 3.79 (dd, J=14.26, 6.50 Hz, 1H), 3.93 (dd, J=14.20, 8.32 Hz, 1H), 4.41 (d. J=5.13 Hz, 1H), 5.33 (dd, J=9.63, 6.00 Hz, 1H), 7.19 (ddd, J=7.69, 4.94, 1.75 Hz, 2H), 7.36-7.48 (m, 8H), 7.86 (ddd, J=7.72, 4.03, 1.75 Hz, 2H), 8.03 (dd, J=7.75, 2.00 Hz, 1H), 8.64 (ddd, J=9.26, 4.88, 1.88 Hz, 2H), 8.70 (dd, J=4.94, 1.81 Hz, 1H).

### Example 46. Preparation of 2-(1-cyano-2,2-diethyl-cyclopropyl)pyridine-3-carbonitrile

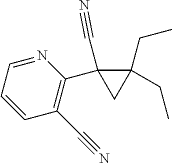 [SMILES: CCC1(CC)CC1(C#N)c1ncccc1C#N]

In a 3 necked round bottom flask equipped with magnetic bar, thermometer and nitrogen inlet was charged DMF (5 mL). To this were added 5-amino-2,2-diethyl-3H-thiophene-4-carbonitrile (0.395 g, 1 equiv., 2.17 mmol) and 2-chloropyridine-3-carbonitrile (0.3 g, 1 equiv., 2.17 mmol) Sodium tert-butanolate (2.2 equiv., 4.76 mmol) was added at room temperature, then the reaction was stirred for two and a half hours at this temperature. 5 mL of ice cold water were added, followed by 5 mL of 2N HCl; the aqueous layer was extracted thrice with 30 mL of EtOAc, the combined organic layers were washed with 20 mL of brine, before being dried over solid Na₂SO₄ and filtered The solvents were removed under reduced pressure to deliver 1.19 g of the crude desired product 2-(1-cyano-2,2-diethyl-cyclopropyl)pyridine-3-carbonitrile as an orange liquid. (Purity measured by Q-NMR being 27% (w/w), the chemical yield is 62%). The crude was purified by chromatography column to give 0.29 g of pure (97% w/w as measured by QNMR), desired product 2-(1-cyano-2,2-diethyl-cyclopropyl)pyridine-3-carbonitrile. Alternatively, the title product can be made from cyclisation of isolated intermediate 2-[2-[(3-cyano-2-pyridyl)sulfanyl]-2-ethyl-butyl]propanedinitrile (example 47) following this protocol. In a 3 necked 25 mL Round bottom flask equipped with magnetic bar, thermometer and nitrogen inlet was charged THF (4 mL). To this was added 2-[2-[(3-cyano-2-pyridyl)sulfanyl]-2-ethyl-butyl]propanedinitrile (180 mg, 1 equiv., 0.63 mmol). Sodium tert-butanolate (1 equiv., 0.63 mmol) was added at room temperature, then the reaction was stirred at this temperature overnight. The reaction mixture was poured onto 2 mL of 2N HCl, stirred for 10 minutes and the aqueous layer was extracted thrice with 20 mL of EtOAc. The combined organic layers were dried over solid Na₂SO₄ and filtered. The solvents were removed under reduced pressure to deliver 0.13 g of the crude desired product 2-(1-cyano-2,2-diethyl-cyclopropyl)pyridine-3-carbonitrile as an orange liquid. (Purity measured by Q-NMR being 53% (w/w), the chemical yield is 48%). The crude was purified by chromatography column to deliver 60 mg of pure 2-(1-cyano-2,2-diethyl-cyclopropyl)pyridine-3-carbonitrile in an isolated yield of 40%. ¹H NMR (CDCl₃, 400 MHz) δ (ppm)=0.52-0.59 (m, 1H), 0.86 (t, J=7.38 Hz, 3H), 1.25 (t, J=7.38 Hz, 3H), 1.37-1.49 (m, 1H), 1.54 (dd, J=5.44, 1.19 Hz, 1H), 1.74 (dq, J=14.81, 7.36 Hz, 1H), 2.05 (dq, J=14.87, 7.51 Hz, 1H), 2.23 (d, J=5.38 Hz, 1H), 7.40 (dd, J=7.82, 4.94 Hz, 1H), 8.03 (dd, J=7.82, 1.81 Hz, 1H), 8.75 (dd, J=4.88, 1.75 Hz, 1H).

### Example 47: Preparation of 2-[2-[(3-cyano-2-pyridyl)sulfanyl]-2-ethyl-butyl]propanedinitrile

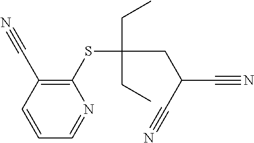 [SMILES: CCC(CC)(CC(C#N)C#N)Sc1ncccc1C#N]

In a 3 necked round bottom flask equipped with magnetic bar, thermometer and nitrogen inlet was charged DMF (5 mL). To this were added 5-amino-2,2-diethyl-3H-thiophene-4-carbonitrile (395 mg, 1 equiv., 2.17 mmol) and 2-chloropyridine-3-carbonitrile (0.3 g, 1 equiv., 2.17 mmol). The reaction mixture was cooled down to −40° C. and Sodium tert-butanolate (2.2 equiv., 4.76 mmol) was added at this temperature. The reaction was stirred 4 hours at this temperature. 5 mL of ice-cold water were added, followed by 5 mL of ice cold 2N HCl; the aqueous layer was extracted thrice with 30 mL of EtOAc, the combined organic layers were washed 20 mL of brine, before being dried over solid Na₂SO₄ and filtered. The solvents were removed under reduced pressure to deliver 1.815 g of the crude desired product 2-[2-[(3-cyano-2-pyridyl)sulfanyl]-2-ethyl-butyl]propanedinitrile as an orange liquid. (Purity measured by Q-NMR being 17% (w/W) the chemical yield is 50%). The crude was purified by chromatography column to give 0.28 g of pure, desired product 2-[2-[(3-cyano-2-pyridyl)sulfanyl]-2-ethyl-butyl]propanedinitrile as a white solid (Purity measured by Q-NMR being 87% (w/w) the isolated yield is 40%). ¹H NMR (CDCl₃, 400 MHz) δ (ppm)=1.01 (t, J=7.40 Hz, 6H), 1.76 (dq, J=14.56, 7.29 Hz, 2H), 1.84-1.96 (m, 2H), 2.81 (d, J=6.24 Hz, 2H), 4.62 (t, J=6.30 Hz, 1H), 7.22-7.30 (m, 1H), 7.91 (dd, J=7.82, 1.83 Hz, 1H), 8.65 (dd, J=4.89, 1.83 Hz, 1H).

### Example 48: Preparation of 2-(1-cyano-2-ethyl-3-heptyl-cyclopropyl)pyridine-3-carbonitrile

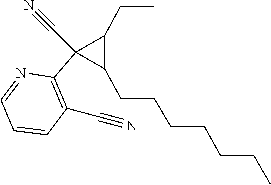 [SMILES: CCCCCCCC1C(CC)C1(C#N)c1ncccc1C#N]

In a 3 necked round bottom flask equipped with magnetic bar, thermometer and nitrogen inlet was charged DMF (5 mL). To this were added 5-amino-3-ethyl-2-heptyl-2,3-dihydrothiophene-4-carbonitrile (0.547 g, 1 equiv., 2.17 mmol) and 2-chloropyridine-3-carbonitrile (0.3 g, 1 equiv., 2.17 mmol). Sodium tert-butanolate (2.2 equiv., 4.76 mmol) was added at room temperature, then the reaction was stirred for five hours at this temperature. 5 mL of ice cold water were added, followed by 5 mL of 2N HCl, the aqueous layer was extracted thrice with 30 mL of EtOAc, the combined organic layers were washed with 20 mL of brine, before being dried over solid Na₂SO₄ and filtered. The solvents were removed under reduced pressure to deliver 1.9 g of the crude desired product 2-(1-cyano-2-ethyl-3-heptyl-cyclopropyl)pyridine-3-carbonitrile as an orange liquid. (Purity measured by Q-NMR being 19% (w/w), the chemical yield is 55%) The crude was purified by chromatography column to give 0.35 g of pure (92% w/w as measured by QNMR), desired product 2-(1-cyano-2-ethyl-3-heptyl-cyclopropyl)pyridine-3-carbonitrile as a white solid. Alternatively, the title product can be made from cyclisation of isolated intermediate 2-[2-[(3-cyano-2-pyridyl)sulfanyl]-1-ethyl-nonyl]propanedinitrile (example 49) following this protocol. In a 3 necked 25 mL Round bottom flask equipped with magnetic bar, thermometer and nitrogen inlet was charged THF (4 mL). To this was added 2-[2-[(3-cyano-2-pyridyl)sulfanyl]-1-ethyl-nonyl]propanedinitrile (250 mg, 1 equiv., 0.71 mmol). Sodium tert-butanolate (1 equiv., 0.71 mmol) was added at room temperature, then the reaction was stirred at this temperature 2 hours. The reaction mixture was poured onto 2 mL of 2N HCl, stirred for 10 minutes and the aqueous layer was extracted thrice with 20 mL of EtOAc. The combined organic layers were dried over solid Na₂SO₄ and filtered. The solvents were removed under reduced pressure to deliver 0.154 g of the crude desired product 2-(1-cyano-2-ethyl-3-heptyl-cyclopropyl)pyridine-3-carbonitrile as an orange liquid. (Purity measured by Q-NMR being 87% (w/w), the chemical yield is 64%). The crude was purified by chromatography column to deliver pure 2-(1-cyano-2-ethyl-3-heptyl-cyclopropyl)pyridine-3-carbonitrile in an isolated yield of 50%. ¹H NMR (CDCl₃, 400 MHz) δ (ppm)=0.83-0.94 (m, 4H), 1.19 (t J=7.40 Hz, 3H), 1.25-1.87 (m, 13H), 1.96-2.17 (m, 2H), 7.36 (dd, J=7.78, 4.77 Hz, 1H), 8.00 (dd, J=7.78, 1.76 Hz, 1H), 8.70 (dd, J=5.02, 1.76 Hz, 1H).

### Example 49: Preparation of 2-[2-[(3-cyano-2-pyridyl)sulfanyl]-1-ethyl-nonyl]propanedinitrile

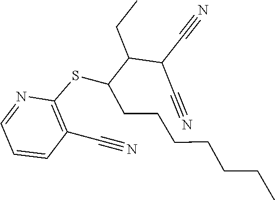 [SMILES: CCCCCCCC(Sc1ncccc1C#N)C(CC)C(C#N)C#N]

In a 3 necked round bottom flask equipped with magnetic bar, thermometer and nitrogen inlet was charged DMF (5 mL). To this were added 5-amino-3-ethyl-2-heptyl-2,3-dihydrothiophene-4-carbonitrile (550 mg, 1 equiv., 2.17 mmol) and 2-chloropyridine-3-carbonitrile (0.3 g, 1 equiv., 2.17 mmol). The reaction mixture was cooled down to −40° C. and Sodium tert-butanolate (2.2 equiv., 4.76 mmol) was added at this temperature. The reaction was stirred 2 hours at this temperature. 5 mL of ice-cold water were added, followed by 5 mL of ice cold 2N HCl; the aqueous layer was extracted thrice with 30 mL of EtOAc, the combined organic layers were washed 20 mL of brine, before being dried over solid Na₂SO₄ and filtered. The solvents were removed under reduced pressure to deliver 0.903 g of the crude desired product 2-[2-[(3-cyano-2-pyridyl)sulfanyl]-1-ethyl-nonyl]propanedinitrile as an orange liquid. (Purity measured by Q-NMR being 46% (w/w) the chemical yield is 54%). The crude was purified by chromatography column to give 0.38 g of pure, desired product 2-[2-[(3-cyano-2-pyridyl)sulfanyl]-1-ethyl-nonyl]propanedinitrile as a white solid (Purity measured by Q-NMR being 93% (w/w) the isolated yield is 46%). ¹H NMR (CDCl₃, 400 MHz) δ (ppm)=0.80-0.96 (m, 4H), 1.10-2.05 (m, 16H), 2.38-2.50 (m, 1H), 4.19-4.41 (m, 2H), 7.16 (ddd, J=7.70, 4.89, 0.73 Hz, 1H), 7.86 (dd, J=7.70, 1.71 Hz, 1H), 8.61 (dt, J=4.89, 1.47 Hz, 1H).

### Example 50: Preparation of 2-(1-cyano-2-ethyl-3-phenyl-cyclopropyl)pyridine-3-carbonitrile

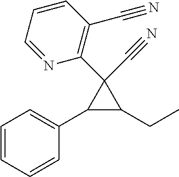 [SMILES: CCC1C(c2ccccc2)C1(C#N)c1ncccc1C#N]

In a 3 necked round bottom flask equipped with magnetic bar, thermometer and nitrogen inlet was charged DMF (5 mL). To this were added a mixture of 5-amino-3-ethyl-2-phenyl-2,3-dihydrothiophene-4-carbonitrile and 5-amino-2-ethyl-3-phenyl-2,3-dihydrothiophene-4-carbonitrile in a 1:2 ratio (500 mg, 1 equiv., 2.17 mmol) and 2-chloropyridine-3-carbonitrile (0.3 g, 1 equiv., 2.17 mmol). Sodium tert-butanolate (2.2 equiv., 4.77 mmol) was added at room temperature, then the reaction was stirred overnight at this temperature. 5 mL of ice cold water were added, followed by 5 mL of 2N HCl; the aqueous layer was extracted thrice with 30 mL of EtOAc, the combined organic layers were washed with 20 mL of brine, before being dried over solid Na₂SO₄ and filtered. The solvents were removed under reduced pressure to deliver 0.72 g of the crude desired product 2-(1-cyano-2-ethyl-3-phenyl-cyclopropyl)pyridine-3-carbonitrile as an orange liquid. (Purity measured by Q-NMR being 56.4% (w/w), the chemical yield is 67%). The crude was purified by chromatography column to give 0.354 g of pure (90% w/was measured by QNMR), desired product 2-(1-cyano-2-ethyl-3-phenyl-cyclopropyl)pyridine-3-carbonitrile as a white solid (in a 8:1 mixture of diastereoisomers) in an isolated yield of 54%; the major isomer NMR being ¹H NMR (CDCl₃, 400 MHz) δ (ppm)=1.25 (t, J=7.4 Hz, 3H), 1.59 (ddd, J=14.6, 8.6, 7.4 Hz, 1H), 1.88 (ddd, J=14.5, 7.3, 5.8 Hz, 1H), 2.43 (td, J=9.1, 5.6 Hz, 1H), 3.37 (d, J=9.5 Hz, 1H), 7.31-7.37 (m, 2H), 7.39-7.48 (m, 3H), 7.56 (d. J=7.6 Hz, 2H), 8.07 (dd, J=7.9, 1.8 Hz, 1H), 8.76 (dd, J=4.9, 1.8 Hz, 1H). Some clear, isolated diagnostic peaks for the minor diastereoisomer are ¹H NMR (CDCl₃, 400 MHz) δ (ppm)=8.82 (dd, J=5.02, 1.76 Hz, 1H) and 3.57 (d, J=8.03 Hz, 1H) Alternatively, the title product can be made from cyclisation of isolated intermediates mixture consisting of 2-[1-[(3-cyano-2-pyridyl)sulfanyl-phenyl-methyl]propyl]propanedinitrile and 2-[2-[(3-cyano-2-pyridyl)sulfanyl]-1-phenyl-butyl]propanedinitrile (example 51) following this protocol. In a 3 necked 25 mL Round bottom flask equipped with magnetic bar, thermometer and nitrogen inlet was charged THF (5 mL). To this was added a mixture consisting of 2-[1-[(3-cyano-2-pyridyl)sulfanyl-phenyl-methyl]propyl]propanedinitrile and 2-[2-[(3-cyano-2-pyridyl)sulfanyl]-1-phenyl-butyl]propanedinitrile in a 1:2 ratio (200 mg, 1 equiv., 0.602 mmol). Sodium tert-butanolate (1 equiv., 0.602 mmol) was added at room temperature, then the reaction was stirred at this temperature during four hours. The reaction mixture was poured onto 2 mL of 2N HCl, stirred for 10 minutes and the aqueous layer was extracted thrice with 20 mL of EtOAc. The combined organic layers were dried over solid Na₂SO₄ and filtered. The solvents were removed under reduced pressure to deliver 0.16 g of the crude desired product 2-(1-cyano-2-ethyl-3-phenyl-cyclopropyl)pyridine-3-carbonitrile as an orange liquid. (Purity measured by Q-NMR being 33% (w/w), the chemical yield is 32%). The crude was purified by chromatography column to give 0.08 g of pure desired product 2-(1-cyano-2-ethyl-3-phenyl-cyclopropyl)pyridine-3-carbonitrile (in a 9.1 mixture of diastereoisomers) giving an isolated yield of 27%

### Example 51: Preparation of 2-[1-[(3-cyano-2-pyridyl)sulfanyl-phenyl-methyl]propyl]propanedinitrile and 2-[2-[(3-cyano-2-pyridyl)sulfanyl]-1-phenyl-butyl]propanedinitrile

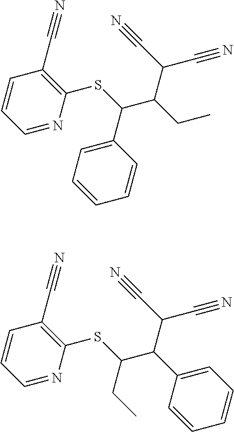 [SMILES: CCC(C(C#N)C#N)C(Sc1ncccc1C#N)c1ccccc1 ; CCC(Sc1ncccc1C#N)C(c1ccccc1)C(C#N)C#N]

In a 3 necked round bottom flask equipped with magnetic bar, thermometer and nitrogen inlet was charged DMF (5 mL). To this were added a mixture of 5-amino-3-ethyl-2-phenyl-2,3-dihydrothiophene-4-carbonitrile and 5-amino-2-ethyl-3-phenyl-2,3-dihydrothiophene-4-carbonitrile in a 1:2 ratio (500 mg, 1 equiv., 2.17 mmol) and 2-chloropyridine-3-carbonitrile (0.3 g, 1 equiv., 2.17 mmol) The reaction mixture was cooled down to −40° C. and Sodium tert-butanolate (2.2 equiv., 3.97 mmol) was added at this temperature. The reaction was stirred 2 hours at this temperature. 5 mL of ice-cold water were added, followed by 10 mL of ice cold 1N HCl; the aqueous layer was extracted thrice with 30 mL of EtOAc, the combined organic layers were washed 20 mL of brine, before being dried over solid Na₂SO₄ and filtered. The solvents were removed under reduced pressure to deliver 2.3 g of the crude desired products 2-[1-[(3-cyano-2-pyridyl)sulfanyl-phenyl-methyl]propyl]propanedinitrile and 2-[2-[(3-cyano-2-pyridyl)sulfanyl]-1-phenyl-butyl]propanedinitrile as an orange liquid. The crude was purified by chromatography column to give a 1.2 mixture of 2-[1-[(3-cyano-2-pyridyl)sulfanyl-phenyl-methyl]propyl]propanedinitrile and 2-[2-[(3-cyano-2-pyridyl)sulfanyl]-1-phenyl-butyl]propanedinitrile with a total isolated yield of 30% and as a pale yellow liquid. ¹H NMR (CDCl₃, 400 MHz) δ (ppm) of the mixture=0.95-1.02 (m, 3H), 1.06 (q, J=7.34 Hz, 2H), 1.63-1.88 (m, 3H), 2.64-2.73 (m, 0.5H), 3.52 (dd, J=11.57, 4.06 Hz, 1H), 3.79 (dd, J=10.38, 4.25 Hz, 0.5H), 4.48 (d, J=3.50 Hz, 0.5H), 4.53 (d, J=10.38 Hz, 0.3H), 4.75 (ddd, J=11.63, 8.57, 3.19 Hz, 1H), 4.80 (d, J=4.13 Hz, 1H), 4.82-4.90 (m, 0.35H), 5.39 (d, J=9.88 Hz, 0.5H), 7.13-7.29 (m, 3H), 7.32-7.56 (m, 10H), 7.83-7.92 (m, 1.5H), 8.66 (dt, J=4.88, 1.31 Hz, 0.7H), 8.68-8.72 (m, 1H).

1. A process for the preparation of a compound of formula (I) 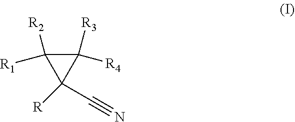
wherein
R is selected from Rₐ or R_{b}, wherein Rₐ is an aryl substituted by at least one electron withdrawing substituent; R_{b} is an unsubstituted or substituted heteroaryl and R₁, R₂, R₃, and R₄ are, Independently from each other, hydrogen, alkyl, aryl, aryloxyakyl or haloaryloxyakyl, and wherein at least one of R₃ or R₄ is hydrogen; which process comprises:
reacting a compound of formula (II) or (III) 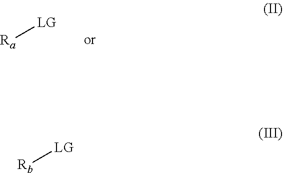
wherein Rₐ and R_{b} are as defined in formula (I); and LG is a halogen or a sulfone group, with a compound of formula (IV), 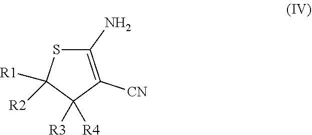
wherein R₁, R₂, R₃, and R₄ are as defined in formula (I);
In the presence of a suitable base, in an appropriate solvent (or diluent);
to produce a compound of formula (I); with the exception of a process for the preparation of 5-(1-cyanocyclopropyl)-pyridine-2-carboxylic acids, esters, amides and nitriles of formula I-b or agrochemically acceptable salts thereof 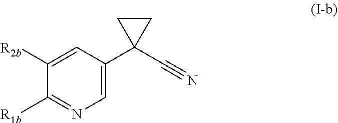
wherein R_{1b} is —CO₂R_{4b}, —CO(NR_{5b}R_{6b}), carboxylate or cyano; R_{2b} is hydrogen, halogen or —SR_{3b}; R_{3b} is C₁-C₄alkyl or C₃-C₆cycloakyl-C₁-C₄alkyl; R_{4b} is hydrogen, —Si(CH₃)₃ or C₁-C₆alkyl; and R_{5b} and R_{6b} are, Independently from each other, hydrogen or C₁-C₄alkyl.

2. The process according to claim 1, wherein Rₐ is a carbocyclic aromatic ring system (such as phenyl or naphthyl) that is mono- or polysubstituted (preferably mono- or di-substituted) by substituents selected from the group consisting of halogen (such as chloro, bromo), cyano, alkyl, haloalkyl, nitro, phenyl, halophenyl, esters, ketones, amides; and wherein at least one such substituent is an electron withdrawing substituent selected from CF₃, NO₂, CN, esters, ketones, and amides.

3. The process according to claim 1, wherein R_{b} is is a five- to ten-membered heteroaromatic ring system that is unsubstituted or is mono- or polysubstituted by substituents selected from the group consisting of halogen, cyano, C₁-C₆alkyl, C₁-C₆haloalkyl, nitro, phenyl and halophenyl; and wherein said ring system can contain 1 or more ring heteroatoms selected from the group consisting of nitrogen, oxygen and sulphur.

4. The process according to calm 1, wherein R₁, R₂, R₃, and R₄ are, independently from each other, hydrogen, C₁-C₇alkyl, phenyl, phenyloxymethyl or halophenyloxymethyl, wherein at least one of R₃ or R₄ is hydrogen.

5. The process according to claim 1, wherein R₁, R₂, R₃, and R₄ are each hydrogen.

6. The process according to claim 1, wherein the suitable base is selected from alkali metal hexamethyldisilazides, alkaline earth metal hexamethyidisilazides, alkali metal hydroxides, alkali metal alkoxides or alkaline earth metal alkoxides.

7. The process according to claim 6, wherein the suitable base is selected from sodium hydroxide, potassium hydroxide, sodium methanolate, sodium tertiobutanolate, and potassium tertiobutanolate; preferably an alkali metal hydroxide, more preferably sodium hydroxide.

8. The process according to claim 1, wherein the solvent (or diluent) is selected from dimethylformamide, dimethylsulfoxide, N-methyl-pyrrolidine, dimethylacetamide, sulfolane and N,N′-dimethylpropyleneurea (DMPU); preferably dimethylformamide, dimethylsulfoxide, or N-methyl-pyrrolidine.

9. The process according to claim 1, which is carried out in a temperature range from approximately 0° C. to approximately +100° C., preferably from approximately +20° C. to approximately +80° C.

10. A compound of formula (INT I) 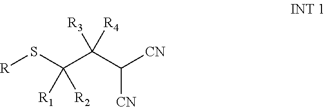
wherein
R is selected from Rₐ or R_{b}, wherein Rₐ is an aryl substituted by at least one electron withdrawing substituent; R_{b} is an unsubstituted or substituted heteroaryl and R₁, R₂, R₃, and R₄ are, Independently from each other, hydrogen, alkyl, aryl, aryloxyalkyl or haloaryloxyalkyl, and wherein at least one of R₃ or R₄ is hydrogen, with the exception of a compound of formula INT b 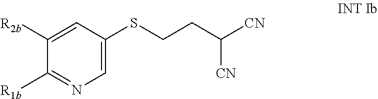
wherein R_{1b} is —CO₂R₄ₐ, —CO(NR_{5b}R_{6b}), carboxylate or cyano; R_{2b} is hydrogen, halogen or —SR_{3b}; R_{3b} is C₁-C₄alkyl or C₃-C₆cycloakyl-C₁-C₄alkyl; R_{4b} is hydrogen, —Si(CH₃)₃ or C₁-C₆alkyl; and R_{5b} and R_{6b} are, Independently from each other, hydrogen or C₁-C₄alkyl.

11. The compound according to claim 10, wherein Rₐ is a carbocyclic aromatic ring system (such as phenyl or naphthyl) that is mono- or polysubstituted (preferably mono- or di-substituted) by substituents selected from the group consisting of halogen (such as chloro, bromo), cyano, alkyl, haloalkyl, nitro, phenyl, halophenyl, esters, ketones, amides; and wherein at least one such substituent is an electron withdrawing substituent selected from CF₃, NO₂, CN, esters, ketones, and amides.

12. The compound according to claim 10, wherein R_{b} is is a five- to ten-membered heteroaromatic ring system that is unsubstituted or is mono- or polysubstituted by substituents selected from the group consisting of halogen, cyano, C₁-C₆alkyl, C₁-C₆haloalkyl, nitro, phenyl and halophenyl; and wherein said ring system can contain 1 or more ring heteroatoms selected from the group consisting of nitrogen, oxygen and sulphur.

13. The compound according to claim 10, wherein R₁, R₂, R₃, and R₄ are, Independently from each other, hydrogen, C₁-C₇alkyl, phenyl, phenyloxymethyl or halophenyloxymethyl, and wherein at least one of R₃ or R₄ is hydrogen.

14. The compound according to claim 10, wherein R₁, R₂, R₃, and R₄ are each hydrogen.
